(19) United States
(12) Patent Application Publication
(10) Pub. No.: US 2022/0267720 A1
(43) Pub. Date: Aug. 25, 2022

(54) FIBROSIS-SPECIFIC CELL CULTURE SUBSTRATE AND METHODS OF USE

(71) Applicant: Xylyx Bio, Inc., Brooklyn, NY (US)

(72) Inventors: John O'NEILL, New York, NY (US); Igal GERMANGUZ, Brooklyn, NY (US); Evelyn ARANDA, Astoria, NY (US); Jennifer XIONG, New York, NY (US); Natalia KISSEL, Brooklyn, NY (US); Alexandra NICHOLS, Brooklyn, NY (US)

(21) Appl. No.: 17/629,046

(22) PCT Filed: Jul. 23, 2020
(86) PCT No.: PCT/US2020/043342

§ 371 Date: Jan. 21, 2022

Related U.S. Application Data
(60) Provisional application No. 62/877,544, filed on Jul. 23, 2019.

(51) Int. Cl. C12N 5/00; G01N 33/50

(52) U.S. Cl. CPC C12N 5/0068; G01N 33/5082; C12N 2500/84; C12N 2533/90; G01N 2800/085; C12N 2501/11; C12N 2501/115

(57) ABSTRACT

An in vitro cell culture substrate is disclosed. The substrate comprises a decellularized tissue-specific extracellular matrix, wherein the tissue-specific extracellular matrix is derived from fibrotic tissue. A method of method of assessing an in vitro fibrotic cell culture is also disclosed. The method comprises providing one or more substrates comprising decellularized tissue-specific extracellular matrix derived from fibrotic tissue, where each substrate is provided in segregated manner. The method further comprises culturing native cells in each substrate to form a fibrotic cell culture. The method further comprises assessing at least one characteristic of each fibrotic cell culture.

### CROSS-REFERENCE TO RELATED APPLICATIONS

This application claims the benefit of priority to U.S. Provisional Application No. 62/877,544 entitled “Fibrosis-Specific Cell Culture Substrate and Methods of Use,” filed Jul. 23, 2019, which is incorporated herein by reference in its entirety.

### GOVERNMENT INTERESTS

This invention was made with government support under Grant No. 1R43HL144341-01A1 awarded by the National Institutes of Health. The Government has certain rights in the invention.

### TECHNICAL FIELD

The present disclosure relates generally to compositions, systems, and methods related to fibrosis-specific extracellular matrix substrates. The disclosed compositions, systems and methods may be utilized, for example, to culture cells in vitro in environments that emulate specific fibrotic niches.

### BACKGROUND

Idiopathic pulmonary fibrosis (IPF) is a chronic interstitial lung disease that primarily affects older adults and is associated with dysregulation of pulmonary fibroblasts, extensive remodeling and deposition of extracellular matrix, and progressive loss of respiratory function. Although advancements have been made in the study and treatment of IPF and drugs have been approved for treatment thereof, the etiology of IPF is still unknown and clinical decline remains common despite treatment. As such, there is a continuing need for research related to IPF and development of new drugs and/or treatments.

A major obstacle to developing safe and effective treatments for IPF is the lack of predictive animal and in vitro models of IPF. While animal models of pulmonary fibrosis are well-established in rodents, the existing models demonstrate a fibrosis that resolves over time unlike the progressive, non-resolving fibrotic process that is characteristic of IPF in humans. Further, the lack of robust, widely adopted in vitro models has hindered predictive basic and translational studies. A major reason for the limited physiologic relevance of these models is that they fail to recapitulate the complexity of the environment present in fibrotic human lungs including the extracellular matrix (ECM).

In its native environment, ECM is a scaffold with tissue-specific cues (e.g., molecular, structural, biomechanical) that provides structure for cell maintenance and growth and mediates cell proliferation, differentiation, gene expression, migration, orientation, and assembly. ECM comprises an interlocking mesh of components including but not limited to viscous proteoglycans (e.g., heparin sulfate, keratin sulfate, and chondroitin sulfate) that provide cushioning, collagen and elastin fibers that provide strength and resilience, and soluble multiadhesive proteins (e.g., fibronectin and laminin) that bind the proteoglycans and collagen fibers to cell receptors. Native extracellular matrix also commonly includes hyaluronic acid and cellular adhesion molecules (CAMs) such as integrins, cadherins, selectins, and immunoglobulins.

The complexity of the ECM has proven difficult to recapitulate in its entirety outside of its native environment. Mimicking just the ECM structure using synthetic biomaterials or mimicking composition by adding purified ECM components is possible. While offering structural mimics, synthetic biomaterials can alter cell behavior (i.e., proliferation, differentiation, gene expression, migration, orientation, and assembly) in vitro and potentially generate cytotoxic by-products at the site of implantation, leading to poor wound healing or an inflammatory environment.

The ECM of each type of tissue may comprises a different composition and properties suited to the tissue's unique set of roles. Further, disease states in tissues may be associated with specific alterations in the biochemical composition, structure, and biomechanics of the ECM environments. For example, the ECM of fibrotic tissue has a different biochemical composition and altered structure and biomechanics as compared to non-fibrotic tissue, which may drive the progression of IPF. Accordingly, IPF models and drug screening platforms that exclude lung-specific ECM and/or fibrosis-specific ECM may lack defining components of the IPF disease environment.

Due to the role of ECM in the progression of IPF and other fibrotic disease, fibrosis-specific ECM (FS-ECM) is a key component for accurately modeling fibrosis and evaluating potential treatments. As such, it would be advantageous to have compositions and tools for in vitro modeling of fibrosis that provide physiologically relevant results by recapitulating the niche environment of fibrotic tissues, e.g., lung FS-ECM and liver FS-ECM.

### SUMMARY

This summary is provided to comply with 37 C.F.R. § 1.73. It is submitted with the understanding that it will not be used to interpret or limit the scope or meaning of the present disclosure.

Embodiments of the invention are directed to an in vitro cell culture substrate comprising a decellularized tissue-specific extracellular matrix, wherein the tissue-specific extracellular matrix is derived from fibrotic tissue.

Embodiments of the invention are directed to a method of assessing an in vitro fibrotic cell culture, the method comprising: providing one or more substrates comprising decellularized tissue-specific extracellular matrix derived from fibrotic tissue, where each substrate is provided in segregated manner; culturing native cells in each substrate to form a fibrotic cell culture; and assessing at least one characteristic of each fibrotic cell culture.

Embodiments of the invention are directed to a method of assessing a drug response of a fibrotic cell culture, the method comprising: providing one or more first substrates comprising decellularized tissue-specific extracellular matrix derived from fibrotic tissue, wherein each first substrate is provided in a segregated manner; providing one or more second substrates comprising decellularized tissue-specific extracellular matrix derived from healthy, non-fibrotic tissue, wherein each second substrate is provided in a segregated manner; culturing native cells in each first substrate to form a fibrotic cell culture; culturing native cells in each first substrate to form a non-fibrotic cell culture; contacting each fibrotic cell culture and each non-fibrotic cell culture with a drug; and assessing a response of each fibrotic cell culture and each non-fibrotic cell culture to the drug.

Embodiments of the present invention are directed to a kit for constructing a plurality of cancer cell culture substrates, the kit comprising: one or more substrate precursors, each substrate precursor comprising a different decellularized tissue-specific extracellular matrix derived from fibrotic tissue; and at least one reagent configured to convert each substrate precursor into a tissue-specific extracellular matrix substrate.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an illustrative diagram of a method of making a fibrosis-specific extracellular matrix substrate in accordance with an embodiment.

FIGS. 2A-2F depict an example of a histological and biochemical characterization of extracellular matrix structural components in idiopathic pulmonary fibrosis and normal lung tissues and matrix scaffolds in accordance with an embodiment.

FIGS. 3A-3J depict an example of a characterization of proteoglycans and glycoproteins in idiopathic pulmonary fibrosis lung tissues and matrix scaffolds in accordance with an embodiment.

FIGS. 4A-4G depict an example of structural, topographical, and mechanical characterizations of idiopathic pulmonary fibrosis lung scaffolds in accordance with an embodiment.

FIGS. 5A-5F depict an example of phenotypes of lung fibroblasts in idiopathic pulmonary fibrosis and normal lung scaffolds in accordance with an embodiment.

FIGS. 6A-6C depicts an example of an approach to produce normal lung- and idiopathic pulmonary fibrosis-specific extracellular matrix substrate for idiopathic fibrosis disease modeling and drug screening platforms in accordance with an embodiment.

FIGS. 7A-7H depict an example of biochemical and mechanical characterization of human normal and idiopathic pulmonary fibrosis lung matrix hydrogels in accordance with an embodiment.

FIGS. 8A-8K depict an example of viability, cytocompatibility, and phenotype of human lung cells in normal lung extracellular matrix hydrogels in accordance with an embodiment.

FIGS. 9A-9J depict an example of histological and biochemical characterization of extracellular matrix components in fibrotic and normal liver tissue and acellular matrix in accordance with an embodiment.

FIGS. 10A-10E depict an example of biocompatibility and comparative function of human hepatocytes in liver extracellular matrix and competing substrates in accordance with an embodiment.

FIGS. 11A-11F depicts an example of biocompatibility, activation and response to ethanol, drug testing of human primary hepatic stellate cells in liver extracellular scaffolds and competing substrates in accordance with an embodiment.

FIGS. 12A-12E depicts an example of histologic and biochemical characterization of extracellular matrix components in fibrotic and normal liver tissue and acellular matrix in accordance with an embodiment.

FIGS. 13A-13J depict an example of characterization of proteoglycans and glycoproteins in fibrotic human liver tissue and matrix in accordance with an embodiment.

FIGS. 14A-14D depict an example of physicomechanical characterization of fibrotic and normal human liver matrix hydrogels in accordance with an embodiment.

FIGS. 15A-15D depict an example of antifibrotic drug testing in idiopathic pulmonary fibrosis scaffolds in accordance with an embodiment.

FIGS. 16A-16C depict an example of an approach to produce normal and fibrotic liver extracellular matrix substrates for liver fibrosis disease modeling and anti-fibrotic drug screening in accordance with an embodiment.

FIGS. 17A-17G depict an example of the biocompatibility and comparative function of human liver cell types in liver ECM scaffolds and competing cell culture substrates in accordance with an embodiment..

FIGS. 18A-18E depict an example of the biocompatibility and comparative function of human liver cell types in liver ECM hydrogel and competing cell culture substrates in accordance with an embodiment.

FIGS. 19A-19D depict an example of the phenotypic changes of primary hepatic stellate cells in fibrotic and normal human liver ECM hydrogels in accordance with an embodiment.

FIGS. 20A-20D depict an example of the drug responses of primary hepatic stellate cells in fibrotic and normal human liver ECM hydrogels in accordance with an embodiment.

FIGS. 21A-21D depict the compatibility of fibrotic human lung ECM hydrogels with analytical techniques and assays used in drug development and high throughput screening in accordance with an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

All percentages, parts and ratios are based upon the total weight of the compositions and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “animal” as used herein includes, but is not limited to, humans and non-human vertebrates such as wild, domestic, and farm animals.

The term “tissue” refers to any aggregation of similarly specialized cells which are united in the performance of a particular function.

The term “disorder” is used in this disclosure to mean, and is used interchangeably with, the terms disease, condition, or illness, unless otherwise indicated.

The terms “administer,” “administering” or “administration” as used herein refer to either directly administering a compound (also referred to as an agent of interest) or pharmaceutically acceptable salt of the compound (agent of interest) or a composition to a subject.

The term “treat,” “treated,” or “treating” as used herein refers to both therapeutic treatment and prophylactic or preventative measures, wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “inhibiting” includes the administration of a compound of the present invention to prevent the onset of the symptoms, alleviating the symptoms, reducing the symptoms, delaying or decreasing the progression of the disease and/or its symptoms, or eliminating the disease, condition or disorder.

As used herein, the term “therapeutic” means an agent utilized to treat, combat, ameliorate, prevent, or improve an unwanted condition or disease of a patient. In part, embodiments of the present invention are directed to the treatment of fibrosis.

In some embodiments, the compounds and methods disclosed herein can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

### Fibrosis-Specific Extracelullar Matrix Substrates

Embodiments of the invention are directed to a substrate for in vitro cell culturing. The substrate comprises a decellularized tissue-specific extracellular matrix derived from fibrotic tissue. The tissue-specific extracellular matrix recapitulates the composition, mechanics, and cell-matrix interactions specific to the particular fibrotic tissue from which it is derived (i.e., fibrosis-specific ECM or FS-ECM). The substrate may be utilized to culture cells to emulate a fibrotic niche environment. In some embodiments, the fibrotic tissue is a tissue exhibiting a particular type or pathology of fibrosis, e.g., idiopathic fibrosis, cystic fibrosis, pulmonary fibrosis, liver fibrosis, steatofibrosis, or cirrhosis.

Fibrotic tissue may comprise tissue having a degree of fibrotic scarring as generally understood in the medical field. For example, fibrotic tissue may include an excessive amount of connective tissue replacing parenchymal tissue or other tissue. In some embodiments, fibrotic tissue may include fibromas. As further described herein, fibrotic tissue may include elevated levels of collagen, reduced levels of elastin, and/or modified level of various components of the extracellular environment as described herein (see, for example, Table 2, Table 3, and Table 4). Fibrosis may be caused by drugs, radiation, environmental factors, autoimmune conditions, and/or occupational factors. In some cases, a cause may not be readily identifiable (i.e., idiopathic). In the case of idiopathic pulmonary fibrosis (IPF), pathological alteration may cause the normal compliant (i.e., rich in elastin) extracellular environment of the lung to shift to an abnormal environment (i.e., rich in fibrillar collagen) that results in the development of a greater quantity of fibroblasts and connective tissue than is normally present in the lung. In some cases, IPF may include lesions such as alveolar lesions.

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

The FS-ECM may be derived from a variety of fibrotic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source may exhibit one or more additional diseases, specific disorders, or health conditions in additional to fibrosis and may be selected for this purpose. The resulting FS-ECM is representative of extracellular matrix from the tissue source, or more generally from tissue having the same relevant characteristics as the tissue source (e.g., juvenile human fibrotic lung tissue will yield lung-specific ECM representative of a juvenile human's lung exhibiting fibrosis).

In some embodiments, the FS-ECM substrate has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, greater than about 10 years, or any individual value or any range between any two values therein.

The FS-ECM may be processed and provided in a variety of substrate formats. In some embodiments, the format of the FS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

In some embodiments, a plurality of ECM substrates may be provided as a cell culture platform. In some embodiments, the cell culture platform comprises at least one FS-ECM substrate and an ECM substrate derived from normal tissue (i.e., healthy, non-fibrotic tissue) of the same or corresponding type. For example, where the FS-ECM substrate is a lung-specific FS-ECM, the cell culture platform may include an ECM substrate derived from normal lung tissue, thereby facilitating study and comparison of the ECM environments. In some embodiments, the cell culture platform comprises a plurality of FS-ECM substrates from different tissue types. For example, the cell culture platform may include lung-specific FS-ECM and liver-specific FS-ECM, thereby facilitating study and comparison of the ECM environments. In some embodiments, the cell culture platform may include a plurality of FS-ECMs from the same tissue type, each FS-ECM being derived from tissue exhibiting a different fibrosis type, pathology, or level of progression. For example, the cell culture platform may include a first FS-ECM derived from lung tissue exhibiting IPF and a second FS-ECM derived from lung tissue exhibiting cystic fibrosis, thereby facilitating study and comparison of the ECM environments. In another example, the cell culture platform may include a first FS-ECM derived from liver tissue exhibiting steatofibrosis and a second FS-ECM derived from liver tissue exhibiting cirrhosis, thereby facilitating study and comparison of the ECM environments as the disease progresses.

In a particular embodiment, the cell culture platform comprises a control ECM substrate derived from normal liver tissue, a first FS-ECM substrate derived from tissue exhibiting steatofibrosis, and a second FS-ECM substrate derived from tissue exhibiting cirrhosis. In another particular embodiment, the cell culture platform comprises a control ECM substrate derived from normal lung tissue and a first FS-ECM substrate derived from lung tissue exhibiting IPF. However, any combination of tissue types, fibrosis types, fibrosis pathologies, fibrosis progression levels, and the like may be represented by the FS-ECMs in the cell culture platform as would be apparent to a person having an ordinary level of skill in the art.

In some embodiments, the cell culture platform may be provided as a cell culture vessel housing the plurality of ECM substrates. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, each ECM substrate of the cell culture platform is segregated, i.e., completely physically separated from other ECM substrates. The physical separation must be capable of preventing cell transfer between the ECM substrates, co-mingling of cell culture components, interaction, cross-contamination, or any other influence of one substrate or culture upon another. In some embodiments, the segregation comprises a barrier such as a wall between the ECM substrates. For example, as described, a tissue culture plate with a plurality of wells may be utilized such that the walls of the wells serve as a physical barrier between the ECMs. Other types of barriers may be utilized as would be known to one having an ordinary level of skill in the art. In some embodiments, an adequate amount of physical spacing between ECM substrates may provide sufficient segregation. For example, as described above, a tissue culture plate may include divided regions which are adequately spaced to provide for individual ECM substrates. Further, in some embodiments, multiple plates or vessels may be utilized, where one or more ECMs are provided on each plate or vessel in order to provide segregation. Various additional manners of providing physical separation between substrates as would be known to one having an ordinary level of skill in the art are contemplated herein.

In additional embodiments, each ECM substrate may be compartmentalized, i.e., physically separated from the other ECM substrates to prevent intermixing in a manner that would substantially alter the composition of any of the ECM substrates. Compartmentalized ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the ECMs and the interconnectivity therebetween may mimic the interconnectivity of parts of an organ, a plurality of organs, and/or an organ system.

The FS-ECM has a specified composition that emulates the ECM found in a specific native fibrotic tissue. As such, the composition of each FS-ECM may vary. Each FS-ECM may comprise ECM scaffolding proteins, ECM-associated proteins, ECM regulators, and secreted factors in the extracellular fluid. The composition described herein may be unique from ECM substrates produced by various conventional methods by the inclusion of these various components. While conventional methods utilize slices or sections of ECM scaffold from natural tissue for cell culturing, the scaffold alone may lack several components found only in the ECF and/or the greater matrisome. Furthermore, the concentrations of various components in the scaffold alone may differ from the concentrations of the same components in the whole tissue (i.e., due to the differing composition of the greater matrisome). For example, Table 2 and Table 3 demonstrate that, in the case of both healthy and fibrotic tissue, the scaffold may have differing concentrations with respect to the whole tissue and/or may lack components detected in the whole tissue. Accordingly, the ECM substrates described herein may process sections of ECM scaffold and tissue in a manner that does not remove or compromise components of the extracellular environment beyond the scaffold. Therefore, the ECM substrates described herein include components beyond that which is found in ECM scaffold in vivo, thereby more accurately emulating the in vivo extracellular environment of the tissue.

Each FS-ECM may comprise a different combination of proteoglycans, collagens, elastins, multiadhesive proteins, hyaluronic acid, CAMs, and additional components. Each of these components may have subtypes, the presence of each of which may vary from one FS-ECM to another FS-ECM. Each FS-ECM may be characterized by the presence or absence of one or more components. Further, the concentration of each component may vary from one FS-ECM to another FS-ECM. These variations result in each FS-ECM having unique physical characteristics, such as architecture and stiffness, and unique cell interaction characteristics, such as gene expression, ECM remodeling, and cell proliferation.

In some embodiments, lung-specific FS-ECM may comprise about 100-400 μg/mL collagens, less than about 25 μg/mL elastins, and greater than about 1 μg/mL glycosaminoglycans. In some embodiments, the lung-specific FS-ECM has an elastic modulus of about 20 kPa. However, the elastic modulus may be about 20 kPa to about 50 kPa, about 50 kPa to about 100 kPa, about 100 kPa to about 200 kPa, greater than about200 kPa, or individual values or ranges therebetween. In some embodiments, the elastic modulus may be similar to the elastic modulus of fibrotic lung tissue.

In some embodiments, the lung-specific FS-ECM comprises collagens including type I α1, type I α2, type I α3, type II α1, type III α1, type IV α1, type IV α2, type IV α3, type IV α4, type IV α5, type V α1, type V α2, type V α3, type VI α1, type VI α2, type VI α3, type VI α5, type VIII α1, type IX α2, type XI α1, type XI α2, type XXI α1, type XVI α1, and/or procollagen α1(V) collagen chains. In some embodiments, the lung-specific FS-ECM comprises proteoglycans including hyaluronan, heparan sulfate, aggrecan core protein, hyaluronan and proteoglycan link protein 1, heparan sulfate proteoglycan 2, and/or heparan sulfate PG core protein. In some embodiments, the lung-specific FS-ECM comprises glycoproteins including dermatopontin, elastin, fibrillin 1, fibrillin 2, fibulin 2, fibulin 5, laminin subunit α (e.g., α3 and/or α5), laminin subunit β (e.g., β2), laminin subunit γ (e.g., γ1), microfibril associated protein 4, nidogen 1, periostin, and/or matrix GLA protein (MGP). In some embodiments, the lung-specific FS-ECM comprises matrisome-secreted factors including hornerin. In some embodiments, the lung-specific FS-ECM comprises ECM regulators including metalloproteinase inhibitor 3, cathepsin G, desmoplakin, serum albumin precursor, α1-antitrypsin, and/or junction plakoglobin. In some embodiments, the lung-specific FS-ECM comprises immune factors including complement component C9, immunoglobulin γ1 heavy chain, serum amyloid P-component, and/or neutrophil defensin 3. In some embodiments, the lung-specific FS-ECM comprises matrix-associated factors including albumin and/or acidic chitinase. In some embodiments, the lung-specific FS-ECM comprises other structural factors including actin γ2, aquaporin-1, and/or keratin structural proteins including type I-cytoskeletal 9, type I-cytoskeletal 10, type I-cytoskeletal 14, type II-cytoskeletal 1, type II-cytoskeletal 2, and/or type II-cytoskeletal 5 keratin structural proteins.

In some embodiments, the lung-specific FS-ECM comprises growth factors including transforming growth factor β3 (TGF-β3), heparin-binding EGF-like growth factor (HB-EGF), basic fibroblast growth factor (bFGF), vascular endothelial growth factor (VEGF), endocrine gland-derived vascular endothelial growth factor (EG-VEGF), growth differentiation factor 15 (GDF-15), insulin-like growth factor binding protein 1 (IGFBP-6), insulin-like growth factor binding protein 6 (IGFBP-6), hepatocyte growth factor (HGF), epidermal growth factor receptor (EGF R), growth differentiation factor 5 (GDF-15), brain-derived neurotrophic factor (BDNF), platelet-derived growth factor AA (PDGF-AA), and/or osteoprotegerin (OPG).

In some embodiments, the composition of the lung-specific FS-ECM may be characterized with respect to ECM derived from normal lung tissue (i.e., healthy, non-fibrotic lung tissue) and/or matrix scaffolds thereof.

In some embodiments, the composition of the lung-specific FS-ECM may be characterized by the presence of one or more components that are absent in normal lung tissue and/or matrix scaffolds thereof. For example, lung-specific FS-ECM may be characterized by the presence of TGF-β3 and/or HB-EGF, which are not present in normal lung tissue. In some embodiments the composition of the lung-specific FS-ECM may be characterized by the absence of one or more components that are present in normal lung tissue and/or matrix scaffolds thereof.

In some embodiments, the composition of the lung-specific FS-ECM may be characterized by an elevated or reduced concentration of one or more components in comparison to normal lung tissue and/or matrix scaffolds thereof. For example, lung-specific FS-ECM may be characterized by elevated level of collagen and/or reduced levels of elastin. In another example, lung-specific FS-ECM may be characterized by elevated levels of type II collagen, type V collagen, type VI collagen, type XVI collagen, and/or specific chains thereof. In another example, lung-specific FS-ECM may be characterized by elevated levels of laminins. In another example, lung-specific FS-ECM may be characterized by elevated levels of fibrillin 2, fibulin 2, MGP, periostin, vitronectin, biglycan, TIMP3, cathepsin G, and/or desmoplakin.

In some embodiments, the composition of the lung-specific FS-ECM may be characterized by a specific concentration value or range for one or more components that is different from normal lung tissue and/or matrix scaffolds thereof. For example, lung-specific FS-ECM may be characterized by a total concentration of collagens above about 100 μg/mL, above about 200 μg/mL, and/or in the range of about 100 μg/mL to about 400 μg/mL. In another example, lung-specific FS-ECM may be characterized by a total concentration of elastins below about 25 μg/mL. In another example, lung-specific FS-ECM may be characterized by a total concentration of glycosaminoglycans above about 1 μg/mg. In another example, lung-specific FS-ECM may be characterized by a total concentration of TGF-β3 above about 10 pg/mL. In another example, lung-specific FS-ECM may be characterized by a total concentration of HB-EGF above about 1 pg/mL. In another example, lung-specific FS-ECM may be characterized by a total concentration of bFGF above about 100 pg/mL. In another example, lung-specific FS-ECM may be characterized by a total concentration of GDF-15 above about 100 pg/mL.

The lung-specific FS-ECM may be characterized by any of the components, concentrations thereof, and/or changes thereof from normal as summarized in Table 1, Table 2, and Table 3. However, these compositions are exemplary in nature and the FS-ECM profiles may vary therefrom as to any number of components. For example, the composition of the substrate may vary from the described concentration values and/or ranges as to any number of components by about 10%, about 20%, about 30%, greater than 30%, or individual values or ranges therebetween.

TABLE 1
Mass spectrometry analysis of IPF lung matrisome. Changes from
normal in the abundance of IPF lung matrisome components.

| | | Change from |
|---|---|---|
| Protein | Description | normal |
| Collagens | type II, α1 chain | 5688.9 |
| | type XVI, α1 chain | 511.1 |
| | type I, α1 chain | 260.0 |
| | type VI, α3 chain | 255.6 |
| | type VIII, α1 chain | 202.3 |
| | type V, α1 chain | 196.2 |
| | type I, α2 chain | 188.2 |
| | type V, α2 chain | 164.3 |
| | type VI, α2 chain | 161.8 |
| | type VI, α1 chain | 156.4 |
| | type I, α3 chain | 139.0 |
| | type V, α3 chain | 127.4 |
| | type IV, α2 chain | −32.9 |
| | type IV, α1 chain | −35.0 |
| | type IV, α3 chain | −63.5 |
| | type IV, α5 chain | −63.7 |
| | type IV, α4 chain | −70.2 |
| | type XXI, α1 chain | −72.9 |
| Glycoproteins | vitronectin | 966.7 |
| | periostin | 295.8 |
| | fibulin 2 | 222.0 |
| | laminin subunit a5 | 169.1 |
| | dermatopontin | 107.7 |
| | laminin subunit β2 | −38.6 |
| | laminin subunit γ1 | −43.9 |
| | nidogen 1 | −52.8 |
| | laminin subunit α3 | −60.0 |
| Proteoglycans | biglycan | 633.3 |
| | heparan sulfate PG core protein | −37.8 |
| | (BM-specific) | |
| Elastin | elastin isoform | −31.1 |
| Matrisome secreted | hornerin | 101.4 |
| factors | | |
| ECM regulators | metalloproteinase inhibitor 3 | 637.5 |
| | (TIMP3) | |
| | cathepsin G | 500 |
| | desmoplakin | 414.3 |
| | serum albumin precursor | 278.8 |
| | α1-antitrypsin | 240 |
| | junction plakglobin | 202.9 |
| Immune factors | complement component C9 | 1422 |
| | immunoglobulin γ1 heavy chain | 688.9 |
| | serum amyloid P-component | 298.7 |
| | neutrophil defensin 3 | −28.1 |
| Keratin structural | type I, cytoskeletal 9 | 259 |
| proteins | type I, cytoskeletal 14 | 170.6 |
| | type II, cytoskeletal 2 | 167.2 |
| | type II, cytoskeletal 5 | 162.4 |
| | type I, cytoskeletal 10 | 149.8 |
| | type I, cytoskeletal 1 | 145.9 |

BM: basement membrane;
PG: proteoglycan.

TABLE 2
Quantification of growth factors in idiopathic pulmonary fibrosis and normal lung tissues.
Growth factor concentrations were measured by multiplex growth factor array.

| | | | | Fold |
|---|---|---|---|---|
| | | Concentration (pg mL⁻¹) | | change |
| Growth | | Normal | IPF | from |
| factor | Description | tissue | tissue | normal |
| TGF-β3 | Transforming growth factor β3 | ND | 65.8 | * |
| HB-EGF | Heparin-binding EGF-like growth factor | ND | 4.2 | * |
| IGFBP-1 | Insulin-like growth factor binding protein 1 | 0.5 | 86.8 | +159.5 |
| bFGF | Basic fibroblast growth factor | 10.1 | 213.8 | +21.2 |
| EG-VEGF | Endocrine gland-derived vascular endothelial | 2.2 | 38.1 | +17.3 |
| | growth factor | | | |
| BDNF | Brain-derived neurotrophic factor | 31.1 | 145.4 | +4.7 |
| GDF-15 | Growth differentiation factor 15 | 97.8 | 243.4 | +2.5 |
| PDGF-AA | Platelet-derived growth factor AA | 228.1 | 486.7 | +2.1 |
| IGFBP-6 | Insulin-like growth factor binding protein 6 | 123.0 | 228.5 | +1.9 |
| HGF | Hepatocyte growth factor | 13313.2 | 19831.9 | +1.5 |
| VEGF | Vascular endothelial growth factor | 135.9 | 158.8 | +1.2 |
| EGF R | Epidermal growth factor receptor | 17343.2 | 12828.8 | −0.7 |
| OPG | Osteoprotegerin | 60.8 | 33.8 | −0.6 |

ND: not detected.
* Idiopathic pulmonary factor-specific growth factor not detected in normal lung tissue.

TABLE 3
Quantification of growth factors in idiopathic pulmonary fibrosis scaffolds.
Growth factor concentrations were measured by multiplex growth factor array.

| | | | | Fold |
|---|---|---|---|---|
| | | Concentration (pg mL⁻¹) | | change |
| Growth | | Normal | IPF | from |
| factor | Description | scaffold | scaffold | normal |
| GDF-15 | Growth differentiation factor 15 | 0.8 | 14.5 | +18.1 |
| BDNF | Brain-derived neurotrophic factor | 6.3 | 48.7 | +7.7 |
| IGFBP-6 | Insulin-like growth factor binding protein 6 | 14.1 | 71.8 | +5.1 |
| HGF | Hepatocyte growth factor | 23.8 | 91.0 | +3.8 |
| EG-VEGF | Endocrine gland-derived vascular endothelial | 0.6 | 1.5 | +2.5 |
| | growth factor | | | |
| bFGF | Basic fibroblast growth factor | 22.6 | 54.8 | +2.4 |
| HB-EGF | Heparin-binding EGF-like growth factor | 1.4 | 2.5 | +1.8 |
| TGF-β3 | Transforming growth factor β3 | 2.8 | 4.0 | +1.4 |
| VEGF | Vascular endothelial growth factor | 4.6 | 3.0 | −0.3 |
| EGF R | Epidermal growth factor receptor | ND | ND | — |

ND: not detected.
*matricryptic growth factor not detected in tissue.

In some embodiments, the lung-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 20 kPa and/or in the range of about 20 kPa to about 200 kPa.

As described herein, the composition of lung-specific FS-ECM may be configured to support human lung fibroblasts and/or additional types of lung cells in vitro. For example, the lung-specific FS-ECM substrate may be configured to support human lung fibroblasts for in vitro testing of pharmaceuticals. Further, the lung-specific FS-ECM substrate may be configured to facilitate growth and proliferation of the human lung fibroblasts in a manner consistent with fibrosis, i.e., inducing the diseased cell phenotype. Accordingly, the FS-ECM substrate may induce gene expression, growth factor secretion, and other characteristics in a manner consistent with fibrosis. However, the lung-specific FS-ECM may be configured to support a variety of additional cell types found in the lung, i.e., native cells.

In some embodiments, liver-specific FS-ECM may comprise about 600-700 μg/mg collagens, less than about 18 μg/mg elastins, and greater than about 10 μg/mg glycosaminoglycans. In some embodiments, the liver-specific FS-ECM has an elastic modulus of about 15 kPa. However, the elastic modulus may be about 15 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic liver tissue.

In some embodiments, the liver-specific FS-ECM comprises collagens type I α1, type I α2, type II α1, type III α1, type IV α1, type IV α2, type V α2, type VI α1, type VI α2, type VI α3, type VI α5, type VI α6, type VIII α1, type XII α1, type XIV α1, and type XVIII α1 collagen chains. In some embodiments, the liver-specific FS-ECM comprises proteoglycans including versican core protein, decorin, lumican, prolargin, biglycan, asporin, mimecan, heparan sulfate, heparan sulfate proteoglycan 2, and/or BM-specific heparan sulfate PG core protein. In some embodiments, the liver-specific FS-ECM comprises glycoproteins including TGF-β3 or transforming growth factor-β-induced, laminin subunit α5, laminin subunit β1, laminin subunit β2, laminin subunit γ1, periostin, fibrillin 1, fibronectin 1, fibrinogen a chain, fibrinogen β chain, fibrinogen γ chain, dermatopontin, nidogen-1, vitronectin, EGF-contained fibulin-like ECM protein, elastin, fibrillin 2, saposin-B-val, prostate stem cell antigen, and/or von Willebrand factor. In some embodiments, the liver-specific FS-ECM comprises ECM regulators including protein glutamine γ-glutamyltransferase 2, serum albumin precursor, and/or metalloproteinase inhibitor 3 (TIMP3). In some embodiments, the liver-specific FS-ECM comprises immune factors including immunoglobin γ-1 heavy chain, immunoglobin heavy constant γ, complement component C3, complement component C9, serum amyloid P-component, and/or C4b-binding protein a chain. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cyto skeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

In some embodiments, the composition of the liver-specific FS-ECM may be characterized with respect to ECM derived from normal liver tissue (i.e., healthy, non-fibrotic liver tissue) and/or matrix scaffolds thereof. In some embodiments the composition of the liver-specific FS-ECM may be characterized by the presence of one or more components that are absent in normal liver tissue and/or matrix scaffolds thereof. In some embodiments the composition of the liver-specific FS-ECM may be characterized by the absence of one or more components that are present in normal liver tissue and/or matrix scaffolds thereof.

In some embodiments the composition of the liver-specific FS-ECM may be characterized by an elevated or reduced concentration of one or more components in comparison to normal liver tissue and/or matrix scaffolds thereof. For example, liver-specific FS-ECM may be characterized by elevated levels of collagen and/or reduced levels of elastin. In another example, liver-specific FS-ECM may be characterized by elevated levels of type I collagen, type VI collagen, type VIII collagen, type XII collagen, type XIV collagen, and/or specific chains thereof.

In some embodiments the composition of the liver-specific FS-ECM may be characterized by a specific concentration value or range for one or more components that is different from normal liver tissue and/or matrix scaffolds thereof. For example, lung-specific FS-ECM may be characterized by a total concentration of collagens above about 500 μg/mg, above about 600 μg/mg, and/or in the range of about 500 μg/mg to about 700 μg/mg. In another example, lung-specific FS-ECM may be characterized by a total concentration of elastins below about 18 μg/mg. In another example, lung-specific FS-ECM may be characterized by a total concentration of glycosaminoglycans above about 10 μg/mg.

The liver-specific FS-ECM may be characterized by any of the components, concentrations thereof, and/or changes thereof from normal as summarized in Table 4. However, these compositions are exemplary in nature and the FS-ECM profiles may vary therefrom as to any number of components. For example, the composition of the substrate may vary from the described concentration values and/or ranges as to any number of components by about 10%, about 20%, about 30%, greater than 30%, or individual values or ranges therebetween.

TABLE 4
Mass spectrometry summary analysis of fibrotic human
liver matrisome. Changes from normal in the abundance
of fibrotic liver matrisome components.

| | | | Change from |
|---|---|---|---|
| | Normal | Fibrotic | normal (%) |
| Collagens | | | |
| type XIV, α1 chain | 3.6 | 59.5 | 1625.0 |
| type XII, α1 chain | 6.5 | 24.5 | 376.9 |
| type I, α2 chain | 20.5 | 55.5 | 270.7 |
| type VIII, α1 chain | 4.5 | 10.7 | 234.5 |
| type I, α1 chain | 48.8 | 110.7 | 226.7 |
| type XVIII, α1 chain | 9.6 | 17.1 | 177.5 |
| type VI, α1 chain | 293.2 | 460.2 | 156.9 |
| type III, α1 chain | 80.3 | 124.9 | 155.4 |
| type VI, α3 chain | 653.3 | 950.2 | 145.4 |
| type VI, α2 chain | 247.1 | 314.3 | 127.1 |
| type IV, α2 chain | 162.0 | 188.1 | 116.1 |
| type IV, α1 chain | 68.5 | 65.6 | −95.8 |
| type VI, α6 chain | 16.0 | 11.0 | −69.2 |
| Glycoproteins | | | |
| transforming growth factor-β-induced | 1.4 | 38.2 | 2700.0 |
| laminin subunit β1 | 1.0 | 24.3 | 2246.1 |
| periostin | 1.0 | 21.5 | 2150.0 |
| fibrillin-1 | 6.75 | 48.0 | 712.3 |
| fibronectin 1 | 6.0 | 23.2 | 382.1 |
| laminin subunit β2 | 12.3 | 36.0 | 292.5 |
| laminin subunit γ1 | 18.5 | 53.0 | 286.4 |
| fibrinogen β chain | 7.0 | 18.9 | 270.2 |
| dermatopontin | 7.6 | 20.5 | 268.4 |
| nidogen-1 | 4.5 | 11.5 | 255.5 |
| fibrinogen γ chain | 7.4 | 18.4 | 248.3 |
| laminin subunit α5 | 25.8 | 61.9 | 239.6 |
| fibrinogen α chain | 7.3 | 14.2 | 190.9 |
| vitronectin | 28.2 | 33.5 | 118.8 |
| Proteoglycans | | | |
| versican core protein | 1.0 | 20.0 | 2000.0 |
| decorin | 5.5 | 69.3 | 1260.6 |
| lumican | 4.4 | 54.8 | 1241.5 |
| prolargin | 7.4 | 78.7 | 1061.7 |
| biglycan | 13.6 | 95.8 | 701.2 |
| asporin | 9.5 | 46.9 | 489.5 |
| mimecan | 8.0 | 32.5 | 403.0 |
| BM-specific heparan sulfate PG core | 88.5 | 193.7 | 218.9 |
| protein | | | |
| ECM regulators | | | |
| protein glutamine | 11.5 | 58.3 | 503.5 |
| γ-glutamyltransferase 2 | | | |
| serum albumin precursor | 77.0 | 81.8 | 106.2 |
| metalloproteinase inhibitor 3 (TIMP3) | 9.5 | 4.5 | −48.2 |
| Immune factors | | | |
| immunoglobin γ-1 heavy chain | 20.7 | 44.8 | 216.0 |
| complement C3 | 21.4 | 44.3 | 207.0 |
| immunoglobulin heavy constant γ | 10.9 | 18.2 | 167.1 |
| serum amyloid P-component | 20.4 | 24.0 | 117.5 |
| complement component C9 | 25.2 | 21.0 | −83.4 |
| C4b-binding protein α chain | 14.8 | 8.3 | −56.1 |

BM: basement membrane
PG: proteoglycan.

In some embodiments, the liver-specific FS-ECM substrate may be characterized by additional properties or functions of the substrate. In some embodiments, the liver-specific FS-ECM substrate may be characterized by an elevated mechanical stiffness and/or elastic modulus. For example, the liver-specific FS-ECM substrate may be characterized by an elastic modulus above about 15 kPa and/or in the range of about 15 kPa to about 200 kPa.

As described herein, the composition of liver-specific FS-ECM may be configured to support human hepatic stellate cells and/or additional types of liver cells in vitro. For example, the liver-specific FS-ECM substrate may be configured to support human hepatic stellate cells for in vitro testing of pharmaceuticals. Further, the liver-specific FS-ECM substrate may be configured to facilitate growth and proliferation of the human hepatic stellate cells in a manner consistent with fibrosis, i.e., inducing the diseased cell phenotype. Accordingly, the FS-ECM substrate may induce gene expression, growth factor secretion, and other characteristics in a manner consistent with fibrosis. However, the liver-specific FS-ECM may be configured to support a variety of additional cell types, including but not limited to primary hepatocytes, Hep2G cells, Kupffer cells, sinusoidal endothelial cells, and/or additional cell types found in the liver, i.e., native cells.

In some embodiments, the FS-ECM substrate may further include additional components beyond the FS-ECM components. In some embodiments, the FS-ECM substrate may include components found in the extracellular fluid of fibrotic tissue. For example, a component present in extracellular fluid of fibrotic tissue may not be present in the ECM scaffold thereof and may thus be added to the FS-ECM to further emulate the fibrotic niche environment. In some embodiments, the substrates may include cell culture media, media supplements, or components thereof. In some embodiments, the substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The FS-ECM substrate may further be configured, adapted, made and/or used in any manner described herein with respect to the method of making the FS-ECM substrate, the kit for forming the FS-ECM substrate, and the method of using the FS-ECM substrate.

### Kit for Forming the Substrate Described Herein

In another aspect of the present subject matter, a kit forming a FS-ECM substrate is provided. The kit includes at least one substrate precursor and at least one reagent. Each substrate precursor comprises a decellularized FS-ECM in a form configured to be converted into a FS-ECM substrate. The reagent is adapted to convert the precursor into a FS-ECM substrate.

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

In some embodiments the kit comprises a plurality of substrate precursors. Each substrate precursor may comprise a different decellularized tissue-specific ECM in order to emulate multiple niche environments with a single kit. In some embodiments, a kit may include one or more control precursors comprising a tissue-specific ECM derived from normal tissue, one or more first precursors comprising a first FS-ECM, and one or more second precursors comprising a second FS-ECM. In some embodiments, the kit may include a plurality of FS-ECMs from the same tissue type, each FS-ECM being derived from tissue exhibiting a different fibrosis type, pathology, or level of progression., thereby facilitating study and comparison of the ECM environments. While a combination of two or three different ECM substrates is demonstrated, it should be understood that other quantities are contemplated. A kit may comprise precursors for one, two, three, four, five, or more different ECM substrates.

In a particular embodiment, the kit comprises a first (control) precursor derived from normal liver tissue, a second precursor derived from liver tissue exhibiting steatofibrosis, and a third precursor derived from liver tissue exhibiting cirrhosis. In another particular embodiment, kit comprises a first precursor derived from lung tissue exhibiting IPF and a second precursor derived from lung tissue exhibiting cystic fibrosis. However, any combination of tissue types, fibrosis types, fibrosis pathologies, fibrosis progression levels, and the like may be represented by the FS-ECMs in the cell culture platform as would be apparent to a person having an ordinary level of skill in the art.

In some embodiments, the reagent comprises one or more of a neutral buffer, a basic buffer, a base, and an acid. For example, a neutral buffer may comprise Phosphate Buffered Saline (PBS), TAPSO (3-[N-tris(hydroxymethyl)methylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), and/or MOPS (3-(N-morpholino)propanesulfonic acid). For example, a basic buffer may comprise carbonate bicarbonate, TAPS ([tris(hydroxymethyl)methylamino]propanesulfonic acid), Bicine (2-(bis(2-hydroxyethyl)amino)acetic acid), Tris (tris(hydroxymethyl)aminomethane), and/or Tricine (N-[tris(hydroxymethyl)methyl]glycine). For example, a base may comprise Sodium Hydroxide (NaOH). For example, an acid may comprise Hydrochloric Acid (HCl) or Acetic Acid. In additional embodiments, the reagent may comprise deionized water. However, additional or alternative reagents may be provided to convert the precursor into various forms, as would be known to a person having an ordinary level of skill in the art. In still additional embodiments, a reagent is not required. As such, it may not be provided with the kit. Even further, where a reagent is required, in some embodiments the reagent may nonetheless not be provided with the kit. Rather, the kit may include instructions or indications related to the reagent to be utilized with the substrate precursor. A user may obtain the reagent and utilize it with the kit. For example, a kit may include a substrate precursor and instructions that instruct the user to add deionized water as a reagent. The instructions are described in greater detail below.

The substrate precursor may be provided in a variety of forms. For example, the substrate precursor may be selected from a solution, a dry foam, an intact scaffold, and a dry powder. Additionally, the reagent may be selected to convert the substrate precursor to any of a variety of substrate formats. In some embodiments, the reagent is configured to reconstitute the precursor into a hydrogel. For example, the precursor may comprise a solution and the reagent may comprise a base and a neutral buffer configured to convert the solution into a hydrogel. In another example, the precursor may comprise a dry foam (e.g., a dehydrated or “instant” hydrogel) and the reagent may comprise deionized water and/or a neutral buffer (e.g., PBS, HEPES, and/or TES). In some embodiments, the reagent is configured to reconstitute the precursor into a scaffold. For example, the precursor may comprise a dehydrated scaffold and the reagent may comprise deionized water and/or a neutral buffer (e.g., PBS) configured to rehydrate the scaffold. In another example, the precursor may comprise an intact (hydrated) scaffold and no reagent may be required. In some embodiments, the reagent is configured to solubilize the precursor into a surface coating. For example, the precursor may comprise a solution and the reagent may comprise a basic buffer and/or a neutral buffer configured to convert the solution into a surface coating. In some embodiments, the reagent is configured to convert the precursor into a bio-ink additive. For example, the precursor may comprise a dry powder and the reagent may comprise an acid configured to convert the dry powder into a bio-ink additive. In some embodiments, the reagent is configured to convert the precursor into a media supplement or other liquid solution. For example, the precursor may comprise an acidic solution and the reagent may comprise a neutral buffer (e.g., PBS) configured to neutralize the solution to form a media supplement. In another example, the reagent may comprise a neutral or basic solution and no reagent may be required.

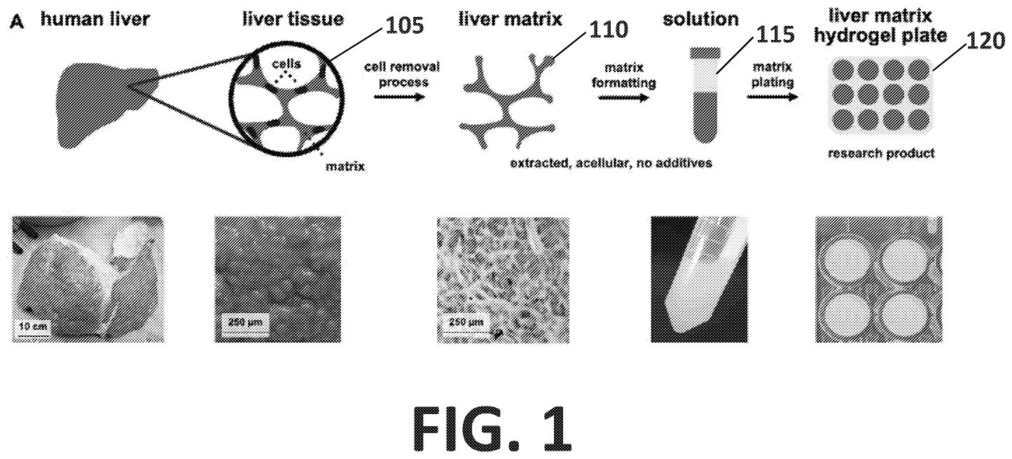

In some embodiments, the one or more precursors of the kit may be prepared by performing the steps of providing 105 one or more tissues, processing 110 the tissue to isolate ECM, and solubilizing 115 the ECM to produce matrix precursors. These steps are more fully described with respect to the method of making a substrate as described herein and depicted in FIG. 1.

According to an exemplary method, tissue is procured and immediately frozen and prepared for sectioning. Frozen blocks are then sectioned longitudinally into thin (about 200 μm-1 mm) slices showing the entire cross-section of the tissue. Portions of the tissue may be dissected and separated from the thin slices prior to decellularization. The tissues are treated using a sequence of chemical, detergent, and enzymatic washes. Each wash is followed by de-ionized water washes. In some embodiments, each region is decellularized by serial washes up to about 12 hours followed by enzymatic digestions. Following decellularization, the ECMs are snap frozen in liquid nitrogen, pulverized, and then lyophilized to obtain a fine powder. Lyophilized ECM powder is digested using an enzymatic agent. The resulting material may be re-constituted into a hydrogel by adding a reagent such as a buffer to adjust the ionic strength and the pH of the solution and forming the FS-ECM substrates.

The described process may be modified or adapted for various tissues described herein. Tissue sections are decellularized by the introduction of one or more of deionized water, hypertonic salines, enzymes, detergents, and acids. In an exemplary embodiment, lobar liver sections are decellularized using a sequence of chemical, detergent, and enzymatic washes. Each wash may be followed by de-ionized water washes.

Following decellularization, resulting materials are terminally sterilized and biopsied according to desired scaffold size. In some embodiments, the scaffold is sized to fit in a cell culture vessel such as the wells of a standard microtiter plate, for example a 6-, 12-, 24-, 48-, or 96-well plate.

In some embodiments, following decellularization, an ECM solution is produced. The decellularized material is snap frozen in liquid nitrogen, pulverized, milled, and lyophilized to obtain a fine ECM powder. In some embodiments, the ECM powder is digested using an enzymatic agent for more than about 1 hour at room temperature. The resulting digest is neutralized, frozen, and thawed to obtain ECM solution, i.e., the substrate precursor. However, the substrate precursor may be provided in other formats as described herein. In some embodiments, the ECM powder may be the substrate precursor. In other embodiments, the ECM powder may be additionally or alternatively processed into one of the other precursor formats described herein.

In some embodiments, the process may be further adapted based on the properties of the fibrotic tissue. In some embodiments, the higher content of connective tissue and/or the greater mechanical stiffness presence in fibrotic tissue may require longer digestion than would be required for normal tissue. In some embodiments, the ECM powder is digested with an enzymatic agent for about 1 hour, about 2 hours, about 3 hours, about 4 hours, about 5 hours, greater than about 5 hours, or individual values or ranges therebetween. In some embodiments, tissue with a greater degree or progression of fibrosis may require a longer digestion time.

In some embodiments, the kit further comprises instructions for utilizing the kit to produce the substrate and/or cell culture platform described herein. The instructions may comprise written or printed instructions, images, graphics, symbols, video files, audio files, links or directions for accessing any of the aforementioned, and combinations thereof. In some embodiments, the instructions include instructions for utilizing the precursor to reconstitute the precursor to a specified format. In some embodiments, the instructions include instructions for plating the reconstituted substrate on a cell culture vessel. In some embodiments, the instructions include instructions for applying the reagent to the precursor. For example, where a reagent is not included in kit, the instructions may include a type of reagent and an amount of reagent to be applied to the precursor. In some embodiments, the instructions comprise instructions for a user to carry out the reconstitution and plating 120 steps as depicted in FIG. 1 and described with respect thereto, thereby forming the cell culture platform. In some embodiments, the instructions comprise instructions for seeding and/or culturing cells within the substrates.

In some embodiments, the kit may be utilized with a cell culture vessel. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, the kit may be utilized to form each ECM substrate on the cell culture vessel in a segregated manner, i.e., completely physically separated from other ECM substrates. The physical separation must be capable of preventing cell transfer between the ECM substrates, co-mingling of cell culture components, interaction, cross-contamination, or any other influence of one substrate or culture upon another. In some embodiments, the segregation comprises a barrier such as a wall between the ECM substrates. For example, as described, a tissue culture plate with a plurality of wells may be utilized such that the walls of the wells serve as a physical barrier between the ECMs. Other types of barriers may be utilized as would be known to one having an ordinary level of skill in the art. In some embodiments, an adequate amount of physical spacing between ECM substrates may provide sufficient segregation. For example, as described above, a tissue culture plate may include divided regions which are adequately spaced to provide for individual ECM substrates. Further, in some embodiments, multiple plates or vessels may be utilized, where one or more ECMs are provided on each plate or vessel in order to provide segregation. Various additional manners of providing physical separation between substrates as would be known to one having an ordinary level of skill in the art are contemplated herein.

In additional embodiments, each ECM substrate may be compartmentalized, i.e., physically separated from the other ECM substrates to prevent intermixing in a manner that would substantially alter the composition of any of the ECM substrates. Compartmentalized ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the ECMs and the interconnectivity therebetween may mimic the interconnectivity of parts of an organ, a plurality of organs, and/or an organ system.

In some embodiments, the kit has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, greater than about 10 years, or any individual value or any range between any two values therein.

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

The FS-ECM may be derived from a variety of fibrotic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source may exhibit one or more additional diseases, specific disorders, or health conditions in additional to fibrosis and may be selected for this purpose. The resulting FS-ECM is representative of extracellular matrix from the tissue source, or more generally from tissue having the same relevant characteristics as the tissue source (e.g., juvenile human fibrotic lung tissue will yield lung-specific ECM representative of a juvenile human's lung exhibiting fibrosis).

Each FS-ECM has a specified composition that emulates the ECM found in a specific native fibrotic tissue. As such, the composition of each FS-ECM may vary. Each FS-ECM may comprise ECM scaffolding proteins, ECM-associated proteins, ECM regulators, and secreted factors in the extracellular fluid. The composition described herein may be unique from ECM substrates produced by various conventional methods by the inclusion of these various components. While conventional methods utilize slices or sections of ECM scaffold from natural tissue for cell culturing, the scaffold alone may lack several components found only in the ECF and/or the greater matrisome. Furthermore, the concentrations of various components in the scaffold alone may differ from the concentrations of the same components in the whole tissue (i.e., due to the differing composition of the greater matrisome). For example, Table 2 and Table 3 demonstrate that, in the case of both healthy and fibrotic tissue, the scaffold may have differing concentrations with respect to the whole tissue and/or may lack components detected in the whole tissue. Accordingly, the ECM substrates described herein may process sections of ECM scaffold and tissue in a manner that does not remove or compromise components of the extracellular environment beyond the scaffold. Therefore, the ECM substrates described herein include components beyond that which is found in ECM scaffold in vivo, thereby more accurately emulating the in vivo extracellular environment of the tissue.

Each FS-ECM may comprise a different combination of proteoglycans, collagens, elastins, multiadhesive proteins, hyaluronic acid, CAMs, and additional components. Each of these components may have subtypes, the presence of each of which may vary from one FS-ECM to another FS-ECM. Each FS-ECM may be characterized by the presence or absence of one or more components. Further, the concentration of each component may vary from one FS-ECM to another FS-ECM. These variations result in each FS-ECM having unique physical characteristics, such as architecture and stiffness, and unique cell interaction characteristics, such as gene expression, ECM remodeling, and cell proliferation.

In some embodiments, lung-specific FS-ECM may comprise about 100-400 μg/mL collagens, less than about 25 μg/mL elastins, and greater than about 1 μg/mL glycosaminoglycans. In some embodiments, the lung-specific FS-ECM has an elastic modulus of about 20 kPa. However, the elastic modulus may be about 20 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic lung tissue.

In some embodiments, the lung-specific FS-ECM comprises collagens including type I α1, type I α2, type I α3, type II α1, type III α1, type IV α1, type IV α2, type IV α3, type IV α4, type IV α5, type V α1, type V α2, type V α3, type VI α1, type VI α2, type VI α3, type VI α5, type VIII α1, type IX α2, type XI α1, type XI α2, type XXI α1, type XVI α1, and/or procollagen α1(V) collagen chains. In some embodiments, the lung-specific FS-ECM comprises proteoglycans including hyaluronan, heparan sulfate, aggrecan core protein, hyaluronan and proteoglycan link protein 1, heparan sulfate proteoglycan 2, and/or heparan sulfate PG core protein. In some embodiments, the lung-specific FS-ECM comprises glycoproteins including dermatopontin, elastin, fibrillin 1, fibrillin 2, fibulin 2, fibulin 5, laminin subunit α (e.g., α3 and/or α5), laminin subunit β (e.g., β2), laminin subunit γ (e.g., γ1), microfibril associated protein 4, nidogen 1, periostin, and/or matrix GLA protein (MGP). In some embodiments, the lung-specific FS-ECM comprises matrisome-secreted factors including hornerin. In some embodiments, the lung-specific FS-ECM comprises ECM regulators including metalloproteinase inhibitor 3, cathepsin G, desmoplakin, serum albumin precursor, α1-antitrypsin, and/or junction plakoglobin. In some embodiments, the lung-specific FS-ECM comprises immune factors including complement component C9, immunoglobulin γ1 heavy chain, serum amyloid P-component, and/or neutrophil defensin 3. In some embodiments, the lung-specific FS-ECM comprises matrix-associated factors including albumin and/or acidic chitinase. In some embodiments, the lung-specific FS-ECM comprises other structural factors including actin γ2, aquaporin-1, and/or keratin structural proteins including type I-cytoskeletal 9, type I-cytoskeletal 10, type I-cytoskeletal 14, type II-cytoskeletal 1, type II-cytoskeletal 2, and/or type II-cytoskeletal 5 keratin structural proteins.

In some embodiments, the lung-specific FS-ECM comprises growth factors including transforming growth factor β3 (TGF-β3), heparin-binding EGF-like growth factor (HB-EGF), basic fibroblast growth factor (bFGF), vascular endothelial growth factor (VEGF), endocrine gland-derived vascular endothelial growth factor (EG-VEGF), growth differentiation factor 15 (GDF-15), insulin-like growth factor binding protein 1 (IGFBP-6), insulin-like growth factor binding protein 6 (IGFBP-6), hepatocyte growth factor (HGF), epidermal growth factor receptor (EGF R), growth differentiation factor 5 (GDF-15), brain-derived neurotrophic factor (BDNF), platelet-derived growth factor AA (PDGF-AA), and/or osteoprotegerin (OPG).

In some embodiments, the composition of the lung-specific FS-ECM may be characterized with respect to ECM derived from normal lung tissue (i.e., healthy, non-fibrotic lung tissue) and/or matrix scaffolds thereof.

In some embodiments, the composition of the lung-specific FS-ECM may be characterized by the presence of one or more components that are absent in normal lung tissue and/or matrix scaffolds thereof. For example, lung-specific FS-ECM may be characterized by the presence of TGF-β3 and/or HB-EGF, which are not present in normal lung tissue. In some embodiments the composition of the lung-specific FS-ECM may be characterized by the absence of one or more components that are present in normal lung tissue and/or matrix scaffolds thereof.

In some embodiments, the composition of the lung-specific FS-ECM may be characterized by an elevated or reduced concentration of one or more components in comparison to normal lung tissue and/or matrix scaffolds thereof. For example, lung-specific FS-ECM may be characterized by elevated level of collagen and/or reduced levels of elastin. In another example, lung-specific FS-ECM may be characterized by elevated levels of type II collagen, type V collagen, type VI collagen, type XVI collagen, and/or specific chains thereof. In another example, lung-specific FS-ECM may be characterized by elevated levels of laminins. In another example, lung-specific FS-ECM may be characterized by elevated levels of fibrillin 2, fibulin 2, MGP, periostin, vitronectin, biglycan, TIMP3, cathepsin G, and/or desmoplakin.

In some embodiments, the composition of the lung-specific FS-ECM may be characterized by a specific concentration value or range for one or more components that is different from normal lung tissue and/or matrix scaffolds thereof. For example, lung-specific FS-ECM may be characterized by a total concentration of collagens above about 100 μg/mL, above about 200 μg/mL, and/or in the range of about 100 μg/mL to about 400 μg/mL. In another example, lung-specific FS-ECM may be characterized by a total concentration of elastins below about 25 μg/mL. In another example, lung-specific FS-ECM may be characterized by a total concentration of glycosaminoglycans above about 1 μg/mg. In another example, lung-specific FS-ECM may be characterized by a total concentration of TGF-β3 above about 10 pg/mL. In another example, lung-specific FS-ECM may be characterized by a total concentration of HB-EGF above about 1 pg/mL. In another example, lung-specific FS-ECM may be characterized by a total concentration of bFGF above about 100 pg/mL. In another example, lung-specific FS-ECM may be characterized by a total concentration of GDF-15 above about 100 pg/mL.

The lung-specific FS-ECM may be characterized by any of the components, concentrations thereof, and/or changes thereof from normal as summarized in Table 1, Table 2, and Table 3. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the lung-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 20 kPa and/or in the range of about 20 kPa to about 200 kPa.

As described herein, the composition of lung-specific FS-ECM may be configured to support human lung fibroblasts and/or additional types of lung cells in vitro. For example, the lung-specific FS-ECM substrate may be configured to support human lung fibroblasts for in vitro testing of pharmaceuticals. Further, the lung-specific FS-ECM substrate may be configured to facilitate growth and proliferation of the human lung fibroblasts in a manner consistent with fibrosis, i.e., inducing the diseased cell phenotype. Accordingly, the FS-ECM substrate may induce gene expression, growth factor secretion, and other characteristics in a manner consistent with fibrosis. However, the lung-specific FS-ECM may be configured to support a variety of additional cell types found in the lung, i.e., native cells.

In some embodiments, liver-specific FS-ECM may comprise about 600-700 μg/mg collagens, less than about 18 μg/mg elastins, and greater than about 10 μg/mg glycosaminoglycans. In some embodiments, the liver-specific FS-ECM has an elastic modulus of about 15 kPa. However, the elastic modulus may be about 15 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic liver tissue.

In some embodiments, the liver-specific FS-ECM comprises collagens type I α1, type I α2, type II α1, type III α1, type IV α1, type IV α2, type V α2, type VI α1, type VI α2, type VI α3, type VI α5, type VI α6, type VIII α1, type XII α1, type XIV α1, and type XVIII α1 collagen chains. In some embodiments, the liver-specific FS-ECM comprises proteoglycans including versican core protein, decorin, lumican, prolargin, biglycan, asporin, mimecan, heparan sulfate, heparan sulfate proteoglycan 2, and/or BM-specific heparan sulfate PG core protein. In some embodiments, the liver-specific FS-ECM comprises glycoproteins including TGF-β3 or transforming growth factor-β-induced, laminin subunit α5, laminin subunit β1, laminin subunit β2, laminin subunit γ1, periostin, fibrillin 1, fibronectin 1, fibrinogen α chain, fibrinogen β chain, fibrinogen γ chain, dermatopontin, nidogen-1, vitronectin, EGF-contained fibulin-like ECM protein, elastin, fibrillin 2, saposin-B-val, prostate stem cell antigen, and/or von Willebrand factor. In some embodiments, the liver-specific FS-ECM comprises ECM regulators including protein glutamine γ-glutamyltransferase 2, serum albumin precursor, and/or metalloproteinase inhibitor 3 (TIMP3). In some embodiments, the liver-specific FS-ECM comprises immune factors including immunoglobin γ-1 heavy chain, immunoglobin heavy constant γ, complement component C3, complement component C9, serum amyloid P-component, and/or C4b-binding protein α chain. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cyto skeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

In some embodiments, the composition of the liver-specific FS-ECM may be characterized with respect to ECM derived from normal liver tissue (i.e., healthy, non-fibrotic liver tissue) and/or matrix scaffolds thereof. In some embodiments the composition of the liver-specific FS-ECM may be characterized by the presence of one or more components that are absent in normal liver tissue and/or matrix scaffolds thereof. In some embodiments the composition of the liver-specific FS-ECM may be characterized by the absence of one or more components that are present in normal liver tissue and/or matrix scaffolds thereof.

In some embodiments the composition of the liver-specific FS-ECM may be characterized by an elevated or reduced concentration of one or more components in comparison to normal liver tissue and/or matrix scaffolds thereof. For example, liver-specific FS-ECM may be characterized by elevated levels of collagen and/or reduced levels of elastin. In another example, liver-specific FS-ECM may be characterized by elevated levels of type I collagen, type VI collagen, type VIII collagen, type XII collagen, type XIV collagen, and/or specific chains thereof.

In some embodiments the composition of the liver-specific FS-ECM may be characterized by a specific concentration value or range for one or more components that is different from normal liver tissue and/or matrix scaffolds thereof. For example, lung-specific FS-ECM may be characterized by a total concentration of collagens above about 500 μg/mg, above about 600 μg/mg, and/or in the range of about 500 μg/mg to about 700 μg/mg. In another example, lung-specific FS-ECM may be characterized by a total concentration of elastins below about 18 μg/mg. In another example, lung-specific FS-ECM may be characterized by a total concentration of glycosaminoglycans above about 10 μg/mg.

The liver-specific FS-ECM may be characterized by any of the components, concentrations thereof, and/or changes thereof from normal as summarized in Table 4. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the liver-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 15 kPa and/or in the range of about 15 kPa to about 200 kPa.

As described herein, the composition of liver-specific FS-ECM may be configured to support human hepatic stellate cells and/or additional types of liver cells in vitro. For example, the liver-specific FS-ECM substrate may be configured to support human hepatic stellate cells for in vitro testing of pharmaceuticals. Further, the liver-specific FS-ECM substrate may be configured to facilitate growth and proliferation of the human hepatic stellate cells in a manner consistent with fibrosis, i.e., inducing the diseased cell phenotype. Accordingly, the FS-ECM substrate may induce gene expression, growth factor secretion, and other characteristics in a manner consistent with fibrosis. However, the liver-specific FS-ECM may be configured to support a variety of additional cell types, including but not limited to primary hepatocytes, Hep2G cells, Kupffer cells, sinusoidal endothelial cells, and/or additional cell types found in the liver, i.e., native cells.

In some embodiments, the precursors and/or substrates formed therewith may further include additional components beyond the FS-ECM components. In some embodiments, the precursors and/or substrates may include components found in the extracellular fluid of fibrotic tissue. For example, a component present in extracellular fluid of fibrotic tissue may not be present in the ECM scaffold thereof and may thus be added to the FS-ECM to further emulate the fibrotic niche environment. In some embodiments, the precursors and/or substrates may include cell culture media, media supplements, or components thereof. In some embodiments, the precursors and/or substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The kit for forming a cell culture platform may further be configured, adapted, made and/or used in any manner described herein with respect to the cell culture platform, the method of making the cell culture platform, and the method of using the cell culture platform.

### Method of Making the Substrates Described Herein

In another aspect of the present subject matter, a method of making a fibrosis-specific extracellular matrix substrate is provided. FIG. 1 depicts a diagram of an illustrative method of making a FS-ECM substrate emulating fibrotic liver niche environment in accordance with an embodiment. However, it is understood that a similar process may be utilized on other fibrotic tissues as described herein and/or on normal tissues as described herein to produce ECM substrates emulating the niche environment of the corresponding tissue. As shown in FIG. 1, fibrotic tissue is provided 105 and the fibrotic tissue is processed 110 to isolate a decellularized FS-ECM. The decellularized FS-ECM is solubilized 115 to produce a matrix solution and reconstituted 120 to form a FS-ECM substrate. While the substrates are depicted as being reconstituted 120 on a cell culture vessel, it is understood that the reconstitution 120 may be performed on any desired vessel or surface.

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

According to an exemplary embodiment, tissue is procured and immediately frozen and prepared for sectioning. Frozen blocks are then sectioned longitudinally into thin (200 μm-1 mm) slices showing the entire cross-section of the tissue. Portions of the tissue may be dissected and separated from the thin slices prior to decellularization. The tissues are treated using a sequence of chemical, detergent, and enzymatic washes. Each wash is followed by de-ionized water washes. In some embodiments, each region is decellularized by serial washes up to 12 hours followed by enzymatic digestions. Following decellularization, the ECMs are snap frozen in liquid nitrogen, pulverized, and then lyophilized to obtain a fine powder. Lyophilized ECM powder is digested using an enzymatic agent. The resulting material may be re-constituted into a hydrogel by adding a reagent such as a buffer to adjust the ionic strength and the pH of the solution and forming the FS-ECM substrates.

The described process may be modified or adapted for various tissues described herein. Tissue sections are decellularized by the introduction of one or more of deionized water, hypertonic salines, enzymes, detergents, and acids. In an exemplary embodiment, lobar liver sections are decellularized by using a sequence of chemical, detergent, and enzymatic washes. Each wash may be followed by de-ionized water washes.

Following decellularization, resulting materials are terminally sterilized and biopsied according to desired scaffold size. In some embodiments, the scaffold is sized to fit in a cell culture vessel such as the wells of a standard microtiter plate, for example a 6-, 12-, 24-, 48-, or 96-well plate.

In some embodiments, following decellularization, an ECM solution is produced. The decellularized material is snap frozen in liquid nitrogen, pulverized, milled, and lyophilized to obtain a fine ECM powder. In some embodiments, the ECM powder is digested using an enzymatic agent for more than about 1 hour at room temperature. The resulting digest is neutralized, frozen, and thawed to obtain ECM solution.

In some embodiments, the process may be further adapted based on the properties of the fibrotic tissue. In some embodiments, the higher content of connective tissue and/or the greater mechanical stiffness presence in fibrotic tissue may require longer digestion than would be required for normal tissue. In some embodiments, to form a solution as described, the ECM powder is digested with an enzymatic agent for about 1 hour, about 2 hours, about 3 hours, about 4 hours, about 5 hours, greater than about 5 hours, or individual values or ranges therebetween. In some embodiments, tissue with a greater degree or progression of fibrosis may require a longer digestion time.

In some embodiments, ECM powder is further processed to form an ECM sponge. ECM powder is digested using an enzymatic agent for less than about 24 hours at room temperature. The resulting digest is subjected to repeated cycles of high-speed centrifugation (5,000 rpm) and vortexing. The resulting material is transferred to a mold of desired dimensions and lyophilized. The resulting sponge can be sectioned, re-sized, or rehydrated. In some embodiments, the sponge is sized to fit in the wells of a standard a microtiter plate, for example a 6-, 12-, 24-, 48-, or 96-well plate.

In some embodiments, the process may be further adapted based on the properties of the fibrotic tissue. In some embodiments, the higher content of connective tissue and/or the greater mechanical stiffness presence in fibrotic tissue may require longer digestion than would be required for normal tissue. In some embodiments, to form a sponge as described, the ECM powder is digested with an enzymatic agent for about 1 hour, about 6 hours, about 12 hours, about 18 hours, about 24 hours, about 36 hours, greater than about 36 hours, or individual values or ranges therebetween. In some embodiments, tissue with a greater degree or progression of fibrosis may require a longer digestion time.

In some embodiments, a plurality of ECM substrates may be formed by the manner described herein. In some embodiments, the plurality of ECM substrates may include one or more FS-ECM substrates in order to emulate multiple fibrotic niche environments. Additionally or alternatively, the plurality of ECM substrates may include an ECM substrate derived from normal tissue to emulate the normal niche environment of a specific tissue. In some embodiments, a control substrate comprises a tissue-specific ECM derived from healthy tissue, a first FS-ECM substrate comprise a first FS-ECM, and second FS-ECM substrate comprises a second FS-ECM. In some embodiments, the kit may include a plurality of FS-ECMs from the same tissue type, each FS-ECM being derived from tissue exhibiting a different fibrosis type, pathology, or level of progression, thereby facilitating study and comparison of the ECM environments. While a combination of three different ECM substrates is demonstrated, it should be understood that other quantities are contemplated. A kit may comprise precursors for one, two, three, four, five, or more different ECM substrates.

In a particular embodiment, the a control substrate may be derived from normal liver tissue, a first FS-ECM substrate may be derived from liver tissue exhibiting steatofibrosis, and a second FS-ECM substrate may be derived from liver tissue exhibiting cirrhosis. In another particular embodiment, a first FS-ECM substrate may be derived from lung tissue exhibiting IPF and a second FS-ECM substrate may be derived from lung tissue exhibiting cystic fibrosis. However, any combination of tissue types, fibrosis types, fibrosis pathologies, fibrosis progression levels, and the like may be represented by the FS-ECMs in the cell culture platform as would be apparent to a person having an ordinary level of skill in the art.

In some embodiments, the substrate may be reconstituted on a cell culture vessel. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

Each ECM substrate may be housed on the cell culture vessel in a segregated manner, i.e., completely physically separated from other ECM substrates. The physical separation must be capable of preventing cell transfer between the ECM substrates, co-mingling of cell culture components, interaction, cross-contamination, or any other influence of one substrate or culture upon another. In some embodiments, the segregation comprises a barrier such as a wall between the ECM substrates. For example, as described, a tissue culture plate with a plurality of wells may be utilized such that the walls of the wells serve as a physical barrier between the ECMs. Other types of barriers may be utilized as would be known to one having an ordinary level of skill in the art. In some embodiments, an adequate amount of physical spacing between ECM substrates may provide sufficient segregation. For example, as described above, a tissue culture plate may include divided regions which are adequately spaced to provide for individual ECM substrates. Further, in some embodiments, multiple plates or vessels may be utilized, where one or more ECMs are provided on each plate or vessel in order to provide segregation. Various additional manners of providing physical separation between substrates as would be known to one having an ordinary level of skill in the art are contemplated herein.

In additional embodiments, each ECM substrate may be compartmentalized, i.e., physically separated from the other ECM substrates to prevent intermixing in a manner that would substantially alter the composition of any of the ECM substrates. Compartmentalized ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the ECMs and the interconnectivity therebetween may mimic the interconnectivity of parts of an organ, a plurality of organs, and/or an organ system.

In some embodiments, the FS-ECM substrate has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, greater than about 10 years, or any individual value or any range between any two values therein.

The FS-ECM may be processed and provided in a variety of substrate formats. In some embodiments, the format of the FS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

The FS-ECM may be derived from a variety of fibrotic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source may exhibit one or more additional diseases, specific disorders, or health conditions in additional to fibrosis and may be selected for this purpose. The resulting FS-ECM is representative of extracellular matrix from the tissue source, or more generally from tissue having the same relevant characteristics as the tissue source (e.g., juvenile human fibrotic lung tissue will yield lung-specific ECM representative of a juvenile human's lung exhibiting fibrosis).

Each FS-ECM has a specified composition that emulates the ECM found in a specific native fibrotic tissue. As such, the composition of each FS-ECM may vary. Each FS-ECM may comprise ECM scaffolding proteins, ECM-associated proteins, ECM regulators, and secreted factors in the extracellular fluid. The composition described herein may be unique from ECM substrates produced by various conventional methods by the inclusion of these various components. While conventional methods utilize slices or sections of ECM scaffold from natural tissue for cell culturing, the scaffold alone may lack several components found only in the ECF and/or the greater matrisome. Furthermore, the concentrations of various components in the scaffold alone may differ from the concentrations of the same components in the whole tissue (i.e., due to the differing composition of the greater matrisome). For example, Table 2 and Table 3 demonstrate that, in the case of both healthy and fibrotic tissue, the scaffold may have differing concentrations with respect to the whole tissue and/or may lack components detected in the whole tissue. Accordingly, the ECM substrates described herein may process sections of ECM scaffold and tissue in a manner that does not remove or compromise components of the extracellular environment beyond the scaffold. Therefore, the ECM substrates described herein include components beyond that which is found in ECM scaffold in vivo, thereby more accurately emulating the in vivo extracellular environment of the tissue.

Each FS-ECM may comprise a different combination of proteoglycans, collagens, elastins, multiadhesive proteins, hyaluronic acid, CAMs, and additional components. Each of these components may have subtypes, the presence of each of which may vary from one FS-ECM to another FS-ECM. Each FS-ECM may be characterized by the presence or absence of one or more components. Further, the concentration of each component may vary from one FS-ECM to another FS-ECM. These variations result in each FS-ECM having unique physical characteristics, such as architecture and stiffness, and unique cell interaction characteristics, such as gene expression, ECM remodeling, and cell proliferation.

In some embodiments, lung-specific FS-ECM may comprise about 100-400 μg/mL collagens, less than about 25 μg/mL elastins, and greater than about 1 μg/mL glycosaminoglycans. In some embodiments, the lung-specific FS-ECM has an elastic modulus of about 20 kPa. However, the elastic modulus may be about 20 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic lung tissue.

In some embodiments, the lung-specific FS-ECM comprises collagens including type I α1, type I α2, type I α3, type II α1, type III α1, type IV α1, type IV α2, type IV α3, type IV α4, type IV α5, type V α1, type V α2, type V α3, type VI α1, type VI α2, type VI α3, type VI α5, type VIII α1, type IX α2, type XI α1, type XI α2, type XXI α1, type XVI α1, and/or procollagen α1(V) collagen chains. In some embodiments, the lung-specific FS-ECM comprises proteoglycans including hyaluronan, heparan sulfate, aggrecan core protein, hyaluronan and proteoglycan link protein 1, heparan sulfate proteoglycan 2, and/or heparan sulfate PG core protein. In some embodiments, the lung-specific FS-ECM comprises glycoproteins including dermatopontin, elastin, fibrillin 1, fibrillin 2, fibulin 2, fibulin 5, laminin subunit α (e.g., α3 and/or α5), laminin subunit β (e.g., β2), laminin subunit γ (e.g., γ1), microfibril associated protein 4, nidogen 1, periostin, and/or matrix GLA protein (MGP). In some embodiments, the lung-specific FS-ECM comprises matrisome-secreted factors including hornerin. In some embodiments, the lung-specific FS-ECM comprises ECM regulators including metalloproteinase inhibitor 3, cathepsin G, desmoplakin, serum albumin precursor, α1-antitrypsin, and/or junction plakoglobin. In some embodiments, the lung-specific FS-ECM comprises immune factors including complement component C9, immunoglobulin γ1 heavy chain, serum amyloid P-component, and/or neutrophil defensin 3. In some embodiments, the lung-specific FS-ECM comprises matrix-associated factors including albumin and/or acidic chitinase. In some embodiments, the lung-specific FS-ECM comprises other structural factors including actin γ2, aquaporin-1, and/or keratin structural proteins including type I-cytoskeletal 9, type I-cytoskeletal 10, type I-cytoskeletal 14, type II-cytoskeletal 1, type II-cytoskeletal 2, and/or type II-cytoskeletal 5 keratin structural proteins.

In some embodiments, the lung-specific FS-ECM comprises growth factors including transforming growth factor β3 (TGF-β3), heparin-binding EGF-like growth factor (HB-EGF), basic fibroblast growth factor (bFGF), vascular endothelial growth factor (VEGF), endocrine gland-derived vascular endothelial growth factor (EG-VEGF), growth differentiation factor 15 (GDF-15), insulin-like growth factor binding protein 1 (IGFBP-6), insulin-like growth factor binding protein 6 (IGFBP-6), hepatocyte growth factor (HGF), epidermal growth factor receptor (EGF R), growth differentiation factor 5 (GDF-15), brain-derived neurotrophic factor (BDNF), platelet-derived growth factor AA (PDGF-AA), and/or osteoprotegerin (OPG).

In some embodiments, the composition of the lung-specific FS-ECM may be characterized with respect to ECM derived from normal lung tissue (i.e., healthy, non-fibrotic lung tissue) and/or matrix scaffolds thereof.

In some embodiments, the composition of the lung-specific FS-ECM may be characterized by the presence of one or more components that are absent in normal lung tissue and/or matrix scaffolds thereof. For example, lung-specific FS-ECM may be characterized by the presence of TGF-β3 and/or HB-EGF, which are not present in normal lung tissue. In some embodiments the composition of the lung-specific FS-ECM may be characterized by the absence of one or more components that are present in normal lung tissue and/or matrix scaffolds thereof.

In some embodiments, the composition of the lung-specific FS-ECM may be characterized by an elevated or reduced concentration of one or more components in comparison to normal lung tissue and/or matrix scaffolds thereof. For example, lung-specific FS-ECM may be characterized by elevated level of collagen and/or reduced levels of elastin. In another example, lung-specific FS-ECM may be characterized by elevated levels of type II collagen, type V collagen, type VI collagen, type XVI collagen, and/or specific chains thereof. In another example, lung-specific FS-ECM may be characterized by elevated levels of laminins. In another example, lung-specific FS-ECM may be characterized by elevated levels of fibrillin 2, fibulin 2, MGP, periostin, vitronectin, biglycan, TIMP3, cathepsin G, and/or desmoplakin.

In some embodiments, the composition of the lung-specific FS-ECM may be characterized by a specific concentration value or range for one or more components that is different from normal lung tissue and/or matrix scaffolds thereof. For example, lung-specific FS-ECM may be characterized by a total concentration of collagens above about 100 μg/mL, above about 200 μg/mL, and/or in the range of about 100 μg/mL to about 400 μg/mL. In another example, lung-specific FS-ECM may be characterized by a total concentration of elastins below about 25 μg/mL. In another example, lung-specific FS-ECM may be characterized by a total concentration of glycosaminoglycans above about 1 μg/mg. In another example, lung-specific FS-ECM may be characterized by a total concentration of TGF-β3 above about 10 pg/mL. In another example, lung-specific FS-ECM may be characterized by a total concentration of HB-EGF above about 1 pg/mL. In another example, lung-specific FS-ECM may be characterized by a total concentration of bFGF above about 100 pg/mL. In another example, lung-specific FS-ECM may be characterized by a total concentration of GDF-15 above about 100 pg/mL.

The lung-specific FS-ECM may be characterized by any of the components, concentrations thereof, and/or changes thereof from normal as summarized in Table 1, Table 2, and Table 3. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the lung-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 20 kPa and/or in the range of about 20 kPa to about 200 kPa.

As described herein, the composition of lung-specific FS-ECM may be configured to support human lung fibroblasts and/or additional types of lung cells in vitro. For example, the lung-specific FS-ECM substrate may be configured to support human lung fibroblasts for in vitro testing of pharmaceuticals. Further, the lung-specific FS-ECM substrate may be configured to facilitate growth and proliferation of the human lung fibroblasts in a manner consistent with fibrosis, i.e., inducing the diseased cell phenotype. Accordingly, the FS-ECM substrate may induce gene expression, growth factor secretion, and other characteristics in a manner consistent with fibrosis. However, the lung-specific FS-ECM may be configured to support a variety of additional cell types found in the lung, i.e., native cells.

In some embodiments, liver-specific FS-ECM may comprise about 600-700 μg/mg collagens, less than about 18 μg/mg elastins, and greater than about 10 μg/mg glycosaminoglycans. In some embodiments, the liver-specific FS-ECM has an elastic modulus of about 15 kPa. However, the elastic modulus may be about 15 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic liver tissue.

In some embodiments, the liver-specific FS-ECM comprises collagens type I α1, type I α2, type II α1, type III α1, type IV α1, type IV α2, type V α2, type VI α1, type VI α2, type VI α3, type VI α5, type VI α6, type VIII α1, type XII α1, type XIV α1, and type XVIII α1 collagen chains. In some embodiments, the liver-specific FS-ECM comprises proteoglycans including versican core protein, decorin, lumican, prolargin, biglycan, asporin, mimecan, heparan sulfate, heparan sulfate proteoglycan 2, and/or BM-specific heparan sulfate PG core protein. In some embodiments, the liver-specific FS-ECM comprises glycoproteins including TGF-β3 or transforming growth factor-β-induced, laminin subunit α5, laminin subunit β1, laminin subunit β2, laminin subunit γ1, periostin, fibrillin 1, fibronectin 1, fibrinogen α chain, fibrinogen β chain, fibrinogen γ chain, dermatopontin, nidogen-1, vitronectin, EGF-contained fibulin-like ECM protein, elastin, fibrillin 2, saposin-B-val, prostate stem cell antigen, and/or von Willebrand factor. In some embodiments, the liver-specific FS-ECM comprises ECM regulators including protein glutamine γ-glutamyltransferase 2, serum albumin precursor, and/or metalloproteinase inhibitor 3 (TIMP3). In some embodiments, the liver-specific FS-ECM comprises immune factors including immunoglobin γ-1 heavy chain, immunoglobin heavy constant γ, complement component C3, complement component C9, serum amyloid P-component, and/or C4b-binding protein α chain. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cyto skeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

In some embodiments, the composition of the liver-specific FS-ECM may be characterized with respect to ECM derived from normal liver tissue (i.e., healthy, non-fibrotic liver tissue) and/or matrix scaffolds thereof. In some embodiments the composition of the liver-specific FS-ECM may be characterized by the presence of one or more components that are absent in normal liver tissue and/or matrix scaffolds thereof. In some embodiments the composition of the liver-specific FS-ECM may be characterized by the absence of one or more components that are present in normal liver tissue and/or matrix scaffolds thereof.

In some embodiments the composition of the liver-specific FS-ECM may be characterized by an elevated or reduced concentration of one or more components in comparison to normal liver tissue and/or matrix scaffolds thereof. For example, liver-specific FS-ECM may be characterized by elevated levels of collagen and/or reduced levels of elastin. In another example, liver-specific FS-ECM may be characterized by elevated levels of type I collagen, type VI collagen, type VIII collagen, type XII collagen, type XIV collagen, and/or specific chains thereof.

In some embodiments the composition of the liver-specific FS-ECM may be characterized by a specific concentration value or range for one or more components that is different from normal liver tissue and/or matrix scaffolds thereof. For example, lung-specific FS-ECM may be characterized by a total concentration of collagens above about 500 μg/mg, above about 600 μg/mg, and/or in the range of about 500 μg/mg to about 700 μg/mg. In another example, lung-specific FS-ECM may be characterized by a total concentration of elastins below about 18 μg/mg. In another example, lung-specific FS-ECM may be characterized by a total concentration of glycosaminoglycans above about 10 μg/mg.

The liver-specific FS-ECM may be characterized by any of the components, concentrations thereof, and/or changes thereof from normal as summarized in Table 4. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the liver-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 15 kPa and/or in the range of about 15 kPa to about 200 kPa.

As described herein, the composition of liver-specific FS-ECM may be configured to support human hepatic stellate cells and/or additional types of liver cells in vitro. For example, the liver-specific FS-ECM substrate may be configured to support human hepatic stellate cells for in vitro testing of pharmaceuticals. Further, the liver-specific FS-ECM substrate may be configured to facilitate growth and proliferation of the human hepatic stellate cells in a manner consistent with fibrosis, i.e., inducing the diseased cell phenotype. Accordingly, the FS-ECM substrate may induce gene expression, growth factor secretion, and other characteristics in a manner consistent with fibrosis. However, the liver-specific FS-ECM may be configured to support a variety of additional cell types, including but not limited to primary hepatocytes, Hep2G cells, Kupffer cells, sinusoidal endothelial cells, and/or additional cell types found in the liver, i.e., native cells.

In some embodiments, the FS-ECM substrates may further include additional components beyond the FS-ECM components. In some embodiments, the FS-ECM substrates may include components found in the extracellular fluid of fibrotic tissue. For example, a component present in extracellular fluid of fibrotic tissue may not be present in the ECM scaffold thereof and may thus be added to the FS-ECM to further emulate the fibrotic niche environment. In some embodiments, the FS-ECM substrates may include cell culture media, media supplements, or components thereof. In some embodiments, the FS-ECM substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The method of making a FS-ECM substrate may further be adapted in any manner described herein with respect to the FS-ECM substrate, the kit for forming the FS-ECM substrates, and the method of using the FS-ECM substrate.

### Methods of Using the Substrates Described Herein

In another aspect of the present invention, methods of using the FS-ECM substrate are provided. In some embodiments, the method comprises providing one or more substrates including one or more FS-ECM substrates as described herein. The method may further comprise culturing cells in the one or more substrates. In some embodiments, culturing cells comprising seeding cells within the one or more substrates and proliferating the cells to form one or more cultures. The method may further comprise assessing at least one characteristic of the one of more cultures. As discussed herein, in some embodiments the method comprises providing two or more different FS-ECM substrates to form cultures in multiple different fibrotic niche environments.

In some embodiments, the at least one characteristic comprises gene expression and/or regulation of genes. For example, expression of specific genes by the cells in the substrates may be evaluated by measuring RNA expression. In some embodiments, the at least one characteristic comprises protein expression and/or regulation of protein-encoding genes. For example, expression of proteins-coding genes may be evaluated by measuring RNA expression of a specific protein-encoding gene and/or assessing the presence and concentration of the specific protein. In some embodiments, the at least one characteristic comprises mechanical stiffness (elastic modulus). In some embodiments, the at least one characteristic comprises proliferation or proliferation rate. In some embodiments, the at least one characteristic comprises ECM interactions. In some embodiments, the at least one characteristic comprises cell differentiation characteristics. In some embodiments, the at least one characteristic comprises cell migration. In some embodiments, the at least one characteristic comprises cell invasion. In some embodiments, the at least one characteristic comprises cell metabolism. In some embodiments, the at least one characteristic comprises cell viability.

In some embodiments, the method may further comprise applying a therapy or a potential therapy to the cell cultures. For example, the method may comprise applying a potential fibrosis therapy drug to the one or more FS-ECM substrates and assessing the at least one characteristic in each of the FS-ECM substrates. In some embodiments, applying a therapy to the cell cultures comprises contacting the cell cultures with a drug. In other embodiments, applying a therapy to the cell cultures comprises applying radiation or other therapies as would be known to one having an ordinary level of skill in the art. However, any potential treatment that may serve as a therapeutic or inhibiting treatment for fibrosis may be utilized herein. In such embodiments, assessing at least one characteristic may comprise evaluating the therapy in order to determine efficacy. For example, the therapy may be a known or potential therapy for fibrosis. The results may be indicative of the drug's potential as a candidate for treatment of fibrosis. Further, where multiple different FS-ECM substrates are evaluated, the results may be instructive of the drug's treatment potential specifically with respect to each type of fibrosis and/or level of progression.

In embodiments where the method includes applying a therapy to the colony, efficacy may be evaluated by applying incremental amounts/concentrations of a drug to similar cell cultures in order to evaluate drug efficacy. The efficacy for each amount/concentration of the drug may be compared in order to determine effective doses.

The FS-ECM may be derived from a variety of types of fibrotic tissue, and thus the resulting FS-ECM may additionally be tissue-specific, emulating the niche environment of a particular type of fibrotic tissue. In some embodiments, the FS-ECM may emulate common sites of fibrosis. For example, the FS-ECM may be selected from lung-specific ECM and liver-specific ECM. In additional embodiments, the FS-ECM may be selected from additional niche environments, such as brain-specific ECM, heart-specific extracellular matrix, skin-specific extracellular matrix, intestine-specific extracellular matrix, bone-specific extracellular matrix, and blood vessel-specific extracellular matrix. In still additional embodiments, the FS-ECM may emulate a niche environment specific to another tissue exhibiting fibrosis as would be apparent to a person having an ordinary level of skill in the art. In some embodiments, the FS-ECM may emulate a region of the anatomy, an organ, or a region of an organ.

In some embodiments, the FS-ECM may be further characterized by a particular type of fibrosis and/or a particular pathology exhibited in the tissue from which the FS-ECM is derived. The FS-ECM may be derived from tissues exhibiting a variety of types and/or pathologies of fibrosis and accordingly may exhibit a unique composition, mechanics, and/or cell-matrix interactions specific to the fibrosis type and/or pathology.

For example, lung-specific ECM may be derived from tissue exhibiting a variety of fibrosis types and/or pathologies. In some embodiments, lung-specific ECM derived from tissue exhibiting IPF may emulate the niche environment associated with IPF (i.e., IPF-specific ECM). In some embodiments, lung-specific ECM derived from tissue exhibiting cystic lung fibrosis may emulate the niche environment associated with cystic lung fibrosis.

In another example, liver-specific ECM may be derived from tissue exhibiting a variety of fibrosis types and/or pathologies. In some embodiments, liver-specific ECM derived from tissue exhibiting steatofibrosis may emulate the niche environment associated with steatofibrosis. In some embodiments, liver-specific ECM derived from tissue exhibiting cirrhosis may emulate the niche environment associated with cirrhosis-related fibrosis. In some embodiments, liver-specific ECM derived from tissue exhibiting bridging fibrosis may emulate the niche environment associated with bridging fibrosis.

In some embodiments, the one or more substrates may further comprise a control substrate. The control substrate may comprising ECM derived from a normal tissue that is relevant to the assessment of the fibrotic cell cultures formed in the FS-ECM substrates. For example, where the FS-ECM substrates are derived from fibrotic liver tissue, the control substrate may be derived from normal liver tissue. In the same manner as described herein, cells may be cultured in the control substrate and the cell culture may be assessed. The assessment of the control substrate may provide “baseline” measurements for comparison with assessment data associated with the fibrotic cell cultures.

In some embodiments, the method comprises providing a plurality of FS-ECM substrates. As such, the method may comprise assessing the at least one characteristic in each of the FS-ECM substrates. In some embodiments, the one or more substrates comprise one or more control substrates and one or more FS-ECM substrates. In some embodiments, the cell culture platform comprises a plurality of FS-ECM substrates from different tissue types. For example, the cell culture platform may include lung-specific FS-ECM and liver-specific FS-ECM, thereby facilitating study and comparison of the ECM environments. In some embodiments, the cell culture platform may include a plurality of FS-ECMs from the same tissue type, each FS-ECM being derived from tissue exhibiting a different fibrosis type, pathology, or level of progression. For example, the cell culture platform may include a first FS-ECM derived from lung tissue exhibiting IPF and a second FS-ECM derived from lung tissue exhibiting cystic fibrosis, thereby facilitating study and comparison of the ECM environments. In another example, the cell culture platform may include a first FS-ECM derived from liver tissue exhibiting steatofibrosis and a second FS-ECM derived from liver tissue exhibiting cirrhosis, thereby facilitating study and comparison of the ECM environments as the disease progresses.

In a particular embodiment, the cell culture platform comprises a control substrate derived from normal liver tissue, a first FS-ECM substrate derived from tissue exhibiting steatofibrosis, and a second FS-ECM substrate derived from tissue exhibiting cirrhosis. In another particular embodiment, the cell culture platform comprises a first ECM substrate derived from normal lung tissue and a first ECM substrate derived from lung tissue exhibiting IPF. However, any combination of tissue types, fibrosis types, fibrosis pathologies, fibrosis progression levels, and the like may be represented by the FS-ECMs in the cell culture platform as would be apparent to a person having an ordinary level of skill in the art.

In some embodiments, the cell culture platform may be provided as a cell culture vessel housing the plurality of ECM substrates. In some embodiments, the cell culture vessel comprises a tissue culture plate. In some embodiments, the cell culture vessel may be a petri dish or other dish. In some embodiments, the cell culture vessel comprises a flask. Additional types of cell culture vessel as would be known to one having an ordinary level of skill in the art are also contemplated herein. The cell culture vessel may comprise one or more divided regions to be utilized for individual ECM substrates. For example, a tissue culture plate may comprise one or more wells. In some embodiments, the plate comprises 1 well, 3 wells, 6 wells, 12 wells, 24 wells, 48 wells, 96 wells, 384 wells, greater than 384 wells, or any individual value or any range between any two values therein.

In some embodiments, each ECM substrate of the cell culture platform is segregated, i.e., completely physically separated from other ECM substrates. The physical separation must be capable of preventing cell transfer between the ECM substrates, co-mingling of cell culture components, interaction, cross-contamination, or any other influence of one substrate or culture upon another. In some embodiments, the segregation comprises a barrier such as a wall between the ECM substrates. For example, as described, a tissue culture plate with a plurality of wells may be utilized such that the walls of the wells serve as a physical barrier between the ECMs. Other types of barriers may be utilized as would be known to one having an ordinary level of skill in the art. In some embodiments, an adequate amount of physical spacing between ECM substrates may provide sufficient segregation. For example, as described above, a tissue culture plate may include divided regions which are adequately spaced to provide for individual ECM substrates. Further, in some embodiments, multiple plates or vessels may be utilized, where one or more ECMs are provided on each plate or vessel in order to provide segregation. Various additional manners of providing physical separation between substrates as would be known to one having an ordinary level of skill in the art are contemplated herein.

In additional embodiments, each ECM substrate may be compartmentalized, i.e., physically separated from the other ECM substrates to prevent intermixing in a manner that would substantially alter the composition of any of the ECM substrates. Compartmentalized ECM substrates may include a means of fluid communication therebetween. For example, the compartmentalization may allow for some cell transfer, interaction, or other influence of one substrate or culture upon another (e.g., transfer of some molecules or creation of a gradient therebetween). In some embodiments, the ECM substrates may be housed in physically separated compartments as described above (e.g., connected vessels, connected chambers of a vessel, etc.) except with fluid channels extending between the compartments. In some embodiments, the compartments comprise microfluidic chambers on a vessel such as chip (e.g., an organ-on-a-chip system). In some embodiments, each compartment comprises a printed bio-ink in a region of a vessel such as a chip. Further, the fluid communication between compartments may be formed in a variety of manners. In some embodiments, the compartments communicate via interconnecting channels spanning between the compartments. For example, the channels may be microfluidic channels. In some embodiments, the compartments are separated by a porous membrane that allows fluid communication therebetween. The fluid communication may be configured to allow transport of fluids, molecules, cells, or a combination thereof. Additionally, the fluid communication may be arranged in a variety of manners. In some embodiments, each of the additional compartments directly fluidly communicate with the first compartment in parallel circuit arrangement. For example, the compartments may be arranged in a hub-and-spoke arrangement where the first compartment serves as a central hub having direct fluid communication with each of the radially arranged additional compartments (i.e., spokes). However, the same structural connectivity may be formed with different physical arrangements. In additional embodiments, the first compartment and the additional compartments directly communicate in a series circuit arrangement (i.e., arranged in a chain) such that some additional compartments indirectly communicate with the first compartment (i.e., fluid communication occurs through a directly communicating compartment). Combinations of parallel and series connections are also contemplated herein. In some embodiments, at least one of the additional compartments directly communicate with the first compartment while the remaining additional compartments indirectly communicate with the first compartment. Several layers of interconnectivity may be formed in this manner. In some embodiments, the interconnectivity may mimic a biological system. For example, the ECMs and the interconnectivity therebetween may mimic the interconnectivity of parts of an organ, a plurality of organs, and/or an organ system.

The FS-ECM may be derived from a variety of fibrotic tissue sources. In some embodiments, the tissue source is selected from a human source and an animal source. For example, the tissue may be porcine (i.e., sourced from a pig) or any other animal tissue known to have clinical relevance. In some embodiments, the tissue source is selected from fetal tissue, juvenile tissue, and adult tissue. In some embodiments, the tissue source may exhibit one or more additional diseases, specific disorders, or health conditions in additional to fibrosis and may be selected for this purpose. The resulting FS-ECM is representative of extracellular matrix from the tissue source, or more generally from tissue having the same relevant characteristics as the tissue source (e.g., juvenile human fibrotic lung tissue will yield lung-specific ECM representative of a juvenile human's lung exhibiting fibrosis).

In some embodiments, the FS-ECM substrate has a shelf life of about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, greater than about 10 years, or any individual value or any range between any two values therein.

The FS-ECM may be processed and provided in a variety of substrate formats. In some embodiments, the format of the FS-ECM substrate may be selected from a hydrogel, a scaffold (e.g., an acellular scaffold), a surface coating, a sponge, fibers (e.g., electrospun fibers), liquid solution, media supplement, and bio-ink (e.g., printable bio-ink).

Each FS-ECM has a specified composition that emulates the ECM found in a specific native fibrotic tissue. As such, the composition of each FS-ECM may vary. Each FS-ECM may comprise ECM scaffolding proteins, ECM-associated proteins, ECM regulators, and secreted factors in the extracellular fluid. The composition described herein may be unique from ECM substrates produced by various conventional methods by the inclusion of these various components. While conventional methods utilize slices or sections of ECM scaffold from natural tissue for cell culturing, the scaffold alone may lack several components found only in the ECF and/or the greater matrisome. Furthermore, the concentrations of various components in the scaffold alone may differ from the concentrations of the same components in the whole tissue (i.e., due to the differing composition of the greater matrisome). For example, Table 2 and Table 3 demonstrate that, in the case of both healthy and fibrotic tissue, the scaffold may have differing concentrations with respect to the whole tissue and/or may lack components detected in the whole tissue. Accordingly, the ECM substrates described herein may process sections of ECM scaffold and tissue in a manner that does not remove or compromise components of the extracellular environment beyond the scaffold. Therefore, the ECM substrates described herein include components beyond that which is found in ECM scaffold in vivo, thereby more accurately emulating the in vivo extracellular environment of the tissue.

Each FS-ECM may comprise a different combination of proteoglycans, collagens, elastins, multiadhesive proteins, hyaluronic acid, CAMs, and additional components. Each of these components may have subtypes, the presence of each of which may vary from one FS-ECM to another FS-ECM. Each FS-ECM may be characterized by the presence or absence of one or more components. Further, the concentration of each component may vary from one FS-ECM to another FS-ECM. These variations result in each FS-ECM having unique physical characteristics, such as architecture and stiffness, and unique cell interaction characteristics, such as gene expression, ECM remodeling, and cell proliferation.

In some embodiments, lung-specific FS-ECM may comprise about 100-400 μg/mL collagens, less than about 25 μg/mL elastins, and greater than about 1 μg/mL glycosaminoglycans. In some embodiments, the lung-specific FS-ECM has an elastic modulus of about 20 kPa. However, the elastic modulus may be about 20 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic lung tissue.

In some embodiments, the lung-specific FS-ECM comprises collagens including type I α1, type I α2, type I α3, type II α1, type III α1, type IV α1, type IV α2, type IV α3, type IV α4, type IV α5, type V α1, type V α2, type V α3, type VI α1, type VI α2, type VI α3, type VI α5, type VIII α1, type IX α2, type XI α1, type XI α2, type XXI α1, type XVI α1, and/or procollagen α1(V) collagen chains. In some embodiments, the lung-specific FS-ECM comprises proteoglycans including hyaluronan, heparan sulfate, aggrecan core protein, hyaluronan and proteoglycan link protein 1, heparan sulfate proteoglycan 2, and/or heparan sulfate PG core protein. In some embodiments, the lung-specific FS-ECM comprises glycoproteins including dermatopontin, elastin, fibrillin 1, fibrillin 2, fibulin 2, fibulin 5, laminin subunit α (e.g., α3 and/or α5), laminin subunit β (e.g., β2), laminin subunit γ (e.g., γ1), microfibril associated protein 4, nidogen 1, periostin, and/or matrix GLA protein (MGP). In some embodiments, the lung-specific FS-ECM comprises matrisome-secreted factors including hornerin. In some embodiments, the lung-specific FS-ECM comprises ECM regulators including metalloproteinase inhibitor 3, cathepsin G, desmoplakin, serum albumin precursor, α1-antitrypsin, and/or junction plakoglobin. In some embodiments, the lung-specific FS-ECM comprises immune factors including complement component C9, immunoglobulin γ1 heavy chain, serum amyloid P-component, and/or neutrophil defensin 3. In some embodiments, the lung-specific FS-ECM comprises matrix-associated factors including albumin and/or acidic chitinase. In some embodiments, the lung-specific FS-ECM comprises other structural factors including actin γ2, aquaporin-1, and/or keratin structural proteins including type I-cytoskeletal 9, type I-cytoskeletal 10, type I-cytoskeletal 14, type II-cytoskeletal 1, type II-cytoskeletal 2, and/or type II-cytoskeletal 5 keratin structural proteins.

In some embodiments, the lung-specific FS-ECM comprises growth factors including transforming growth factor β3 (TGF-β3), heparin-binding EGF-like growth factor (HB-EGF), basic fibroblast growth factor (bFGF), vascular endothelial growth factor (VEGF), endocrine gland-derived vascular endothelial growth factor (EG-VEGF), growth differentiation factor 15 (GDF-15), insulin-like growth factor binding protein 1 (IGFBP-6), insulin-like growth factor binding protein 6 (IGFBP-6), hepatocyte growth factor (HGF), epidermal growth factor receptor (EGF R), growth differentiation factor 5 (GDF-15), brain-derived neurotrophic factor (BDNF), platelet-derived growth factor AA (PDGF-AA), and/or osteoprotegerin (OPG).

In some embodiments, the composition of the lung-specific FS-ECM may be characterized with respect to ECM derived from normal lung tissue (i.e., healthy, non-fibrotic lung tissue) and/or matrix scaffolds thereof.

In some embodiments, the composition of the lung-specific FS-ECM may be characterized by the presence of one or more components that are absent in normal lung tissue and/or matrix scaffolds thereof. For example, lung-specific FS-ECM may be characterized by the presence of TGF-β3 and/or HB-EGF, which are not present in normal lung tissue. In some embodiments the composition of the lung-specific FS-ECM may be characterized by the absence of one or more components that are present in normal lung tissue and/or matrix scaffolds thereof.

In some embodiments, the composition of the lung-specific FS-ECM may be characterized by an elevated or reduced concentration of one or more components in comparison to normal lung tissue and/or matrix scaffolds thereof. For example, lung-specific FS-ECM may be characterized by elevated level of collagen and/or reduced levels of elastin. In another example, lung-specific FS-ECM may be characterized by elevated levels of type II collagen, type V collagen, type VI collagen, type XVI collagen, and/or specific chains thereof. In another example, lung-specific FS-ECM may be characterized by elevated levels of laminins. In another example, lung-specific FS-ECM may be characterized by elevated levels of fibrillin 2, fibulin 2, MGP, periostin, vitronectin, biglycan, TIMP3, cathepsin G, and/or desmoplakin.

In some embodiments, the composition of the lung-specific FS-ECM may be characterized by a specific concentration value or range for one or more components that is different from normal lung tissue and/or matrix scaffolds thereof. For example, lung-specific FS-ECM may be characterized by a total concentration of collagens above about 100 μg/mL, above about 200 μg/mL, and/or in the range of about 100 μg/mL to about 400 μg/mL. In another example, lung-specific FS-ECM may be characterized by a total concentration of elastins below about 25 μg/mL. In another example, lung-specific FS-ECM may be characterized by a total concentration of glycosaminoglycans above about 1 μg/mg. In another example, lung-specific FS-ECM may be characterized by a total concentration of TGF-β3 above about 10 pg/mL. In another example, lung-specific FS-ECM may be characterized by a total concentration of HB-EGF above about 1 pg/mL. In another example, lung-specific FS-ECM may be characterized by a total concentration of bFGF above about 100 pg/mL. In another example, lung-specific FS-ECM may be characterized by a total concentration of GDF-15 above about 100 pg/mL.

The lung-specific FS-ECM may be characterized by any of the components, concentrations thereof, and/or changes thereof from normal as summarized in Table 1, Table 2, and Table 3. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the lung-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 20 kPa and/or in the range of about 20 kPa to about 200 kPa.

As described herein, the composition of lung-specific FS-ECM may be configured to support human lung fibroblasts and/or additional types of lung cells in vitro. For example, the lung-specific FS-ECM substrate may be configured to support human lung fibroblasts for in vitro testing of pharmaceuticals. Further, the lung-specific FS-ECM substrate may be configured to facilitate growth and proliferation of the human lung fibroblasts in a manner consistent with fibrosis, i.e., inducing the diseased cell phenotype. Accordingly, the FS-ECM substrate may induce gene expression, growth factor secretion, and other characteristics in a manner consistent with fibrosis. However, the lung-specific FS-ECM may be configured to support a variety of additional cell types found in the lung, i.e., native cells.

In some embodiments, liver-specific FS-ECM may comprise about 600-700 μg/mg collagens, less than about 18 μg/mg elastins, and greater than about 10 μg/mg glycosaminoglycans. In some embodiments, the liver-specific FS-ECM has an elastic modulus of about 15 kPa. However, the elastic modulus may be about 15 kPa to about 50 kPa, about 50 kPa to about 100 kPa, about 100 kPa to about 200 kPa, greater than about 200 kPa, or individual values or ranges therebetween. In some embodiments, the elastic modulus may be similar to the elastic modulus of fibrotic liver tissue.

In some embodiments, the liver-specific FS-ECM comprises collagens type I α1, type I α2, type II α1, type III α1, type IV α1, type IV α2, type V α2, type VI α1, type VI α2, type VI α3, type VI α5, type VI α6, type VIII α1, type XII α1, type XIV α1, and type XVIII α1 collagen chains. In some embodiments, the liver-specific FS-ECM comprises proteoglycans including versican core protein, decorin, lumican, prolargin, biglycan, asporin, mimecan, heparan sulfate, heparan sulfate proteoglycan 2, and/or BM-specific heparan sulfate PG core protein. In some embodiments, the liver-specific FS-ECM comprises glycoproteins including TGF-β3 or transforming growth factor-β-induced, laminin subunit α5, laminin subunit β1, laminin subunit β2, laminin subunit γ1, periostin, fibrillin 1, fibronectin 1, fibrinogen α chain, fibrinogen β chain, fibrinogen γ chain, dermatopontin, nidogen-1, vitronectin, EGF-contained fibulin-like ECM protein, elastin, fibrillin 2, saposin-B-val, prostate stem cell antigen, and/or von Willebrand factor. In some embodiments, the liver-specific FS-ECM comprises ECM regulators including protein glutamine γ-glutamyltransferase 2, serum albumin precursor, and/or metalloproteinase inhibitor 3 (TIMP3). In some embodiments, the liver-specific FS-ECM comprises immune factors including immunoglobin γ-1 heavy chain, immunoglobin heavy constant γ, complement component C3, complement component C9, serum amyloid P-component, and/or C4b-binding protein a chain. In some embodiments, the liver-specific ECM comprises matrix-associated factors including albumin, acidic chitinase, mucin 5AC (oligomeric mucus/gel-forming), collectin-12, mucin 6 (oligomeric mucus/gel-forming), and/or trefoil factor 2. In some embodiments, the liver-specific ECM comprises other structural factors including actin, keratin type II cyto skeletal 1, keratin type I cytoskeletal 10, keratin type II cytoskeletal 2 epidermal, keratin type I cytoskeletal 9, myosin heavy chain 9, and/or tubulin beta chain. In some embodiments, the liver-specific ECM comprises ECM regulators including granulin precursor.

In some embodiments, the composition of the liver-specific FS-ECM may be characterized with respect to ECM derived from normal liver tissue (i.e., healthy, non-fibrotic liver tissue) and/or matrix scaffolds thereof. In some embodiments the composition of the liver-specific FS-ECM may be characterized by the presence of one or more components that are absent in normal liver tissue and/or matrix scaffolds thereof. In some embodiments the composition of the liver-specific FS-ECM may be characterized by the absence of one or more components that are present in normal liver tissue and/or matrix scaffolds thereof.

In some embodiments the composition of the liver-specific FS-ECM may be characterized by an elevated or reduced concentration of one or more components in comparison to normal liver tissue and/or matrix scaffolds thereof. For example, liver-specific FS-ECM may be characterized by elevated levels of collagen and/or reduced levels of elastin. In another example, liver-specific FS-ECM may be characterized by elevated levels of type I collagen, type VI collagen, type VIII collagen, type XII collagen, type XIV collagen, and/or specific chains thereof.

In some embodiments the composition of the liver-specific FS-ECM may be characterized by a specific concentration value or range for one or more components that is different from normal liver tissue and/or matrix scaffolds thereof. For example, lung-specific FS-ECM may be characterized by a total concentration of collagens above about 500 μg/mg, above about 600 μg/mg, and/or in the range of about 500 μg/mg to about 700 μg/mg. In another example, lung-specific FS-ECM may be characterized by a total concentration of elastins below about 18 μg/mg. In another example, lung-specific FS-ECM may be characterized by a total concentration of glycosaminoglycans above about 10 μg/mg.

The liver-specific FS-ECM may be characterized by any of the components, concentrations thereof, and/or changes thereof from normal as summarized in Table 4. However, these compositions are exemplary in nature and the FS-ECM profiles may vary therefrom as to any number of components. For example, the composition of the substrate may vary from the described concentration values and/or ranges by about 10%, about 20%, about 30%, greater than 30%, or individual values or ranges therebetween.

In some embodiments, the liver-specific FS-ECM substrate may be characterized by additional properties or functions of the substrate. In some embodiments, the lung-specific FS-ECM substrate may be characterized by an elevated mechanical stiffness and/or elastic modulus. For example, the lung-specific FS-ECM substrate may be characterized by an elastic modulus above about 15 kPa and/or in the range of about 15 kPa to about 200 kPa.

As described herein, the composition of liver-specific FS-ECM may be configured to support human hepatic stellate cells and/or additional types of liver cells in vitro. For example, the liver-specific FS-ECM substrate may be configured to support human hepatic stellate cells for in vitro testing of pharmaceuticals. Further, the liver-specific FS-ECM substrate may be configured to facilitate growth and proliferation of the human hepatic stellate cells in a manner consistent with fibrosis, i.e., inducing the diseased cell phenotype. Accordingly, the FS-ECM substrate may induce gene expression, growth factor secretion, and other characteristics in a manner consistent with fibrosis. However, the liver-specific FS-ECM may be configured to support a variety of additional cell types, including but not limited to primary hepatocytes, Hep2G cells, Kupffer cells, sinusoidal endothelial cells, and/or additional cell types found in the liver, i.e., native cells.

In some embodiments, the FS-ECM substrates formed therewith may further include additional components beyond the FS-ECM components. In some embodiments, the FS-ECM substrates may include components found in the extracellular fluid of fibrotic tissue. For example, a component present in extracellular fluid of fibrotic tissue may not be present in the ECM scaffold thereof and may thus be added to the FS-ECM to further emulate the fibrotic niche environment. In some embodiments, the FS-ECM substrates may include cell culture media, media supplements, or components thereof. In some embodiments, the FS-ECM substrates may include one or more of amino acids, glucose, salts, vitamins, carbohydrates, proteins, peptides, trace elements, other nutrients, extracts, additives, gases, or organic compounds. Additional components for the proper growth, maintenance and/or modeling of cells as would be known to one having an ordinary level of skill in the art are also contemplated herein.

The method of using FS-ECM substrates may further be adapted in any manner described herein with respect to the FS-ECM substrates, the method of making FS-ECM substrates, and the kit for forming an FS-ECM substrate.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples:

### EXAMPLE 1

### Characterization of Lung and Extracellular Scaffolds

Donor lung tissue with characteristics of idiopathic pulmonary fibrosis (IPF) and healthy lungs were analyzed to confirm that there were no significant differences in age, height, weight, body mass index, or smoking history. An established numerical rubric was used to assess the extent of histomorphologic disruption and fibrosis. Native lung tissues were sectioned and washed with combinations of chemical, detergent, and enzymatic reagents to obtain acellular human lung ECM, which was confirmed by hematoxylin and eosin staining and quantitative DNA assay. Matrix scaffolds from all human lungs were confirmed negative for mycoplasma, bacteria, and fungi, and deemed suitable for use in cell-based studies.

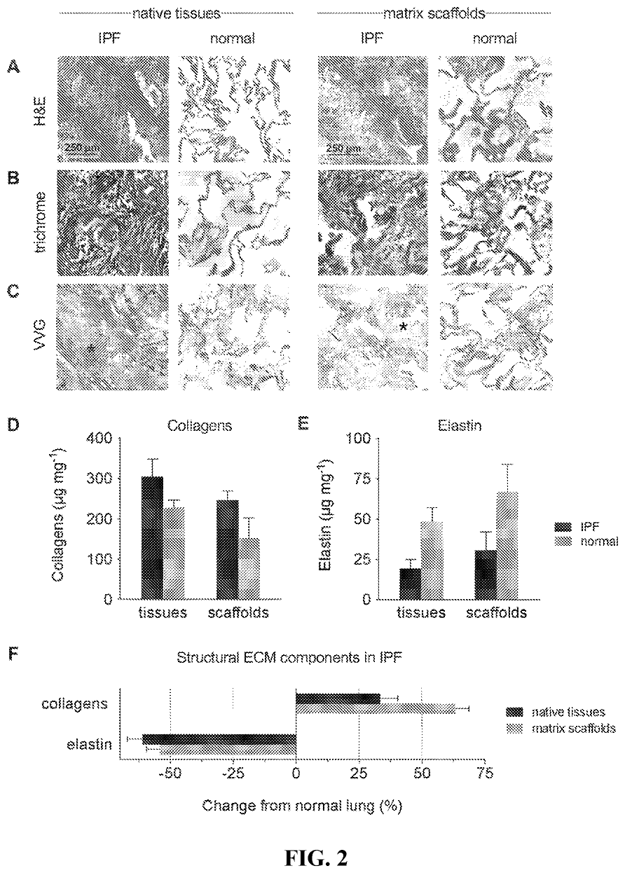

IPF and normal lung tissues and scaffolds were characterized using histology. For histologic evaluations of IPF, representative fields corresponding to fibrosis score 3 (severe fibrosis) were selected. To visualize distributions of ECM structural components in IPF and normal lungs, histologic staining was performed on native (untreated) tissues and matrix scaffolds. H&E staining of native IPF tissues revealed severe distortion of lung structure and large areas of fibrous obliteration with minimal remaining airspace (FIG. 2A). By contrast, H&E staining of native normal lung tissues displayed abundant airspaces defined by thin alveolar septa and stereotypical alveolar saccular architecture. Matrix scaffolds from analogous regions of IPF and normal lungs had no discernible nuclei and displayed drastic differences in scaffold architecture consistent with fibrotic and normal native lung tissues, respectively. Trichrome staining showed dramatic deposition of collagens throughout regions of severe fibrosis (FIG. 2B, arrow indicates normal airway epithelium). In IPF tissues and scaffolds, collagen fibers were observed in densely aligned bundles and in loosely disorganized networks; whereas in normal lung tissues and scaffolds, collagen was organized along alveolar septa and within the interstitium. Verhoeff-Van Gieson (VVG) elastic staining showed a notable loss of elastic fibers (black) in regions of IPF tissues and scaffolds with severe fibrosis, whereas in normal lung tissues and scaffolds elastic fibers were dispersed homogenously throughout the respiratory zone (FIG. 2C).

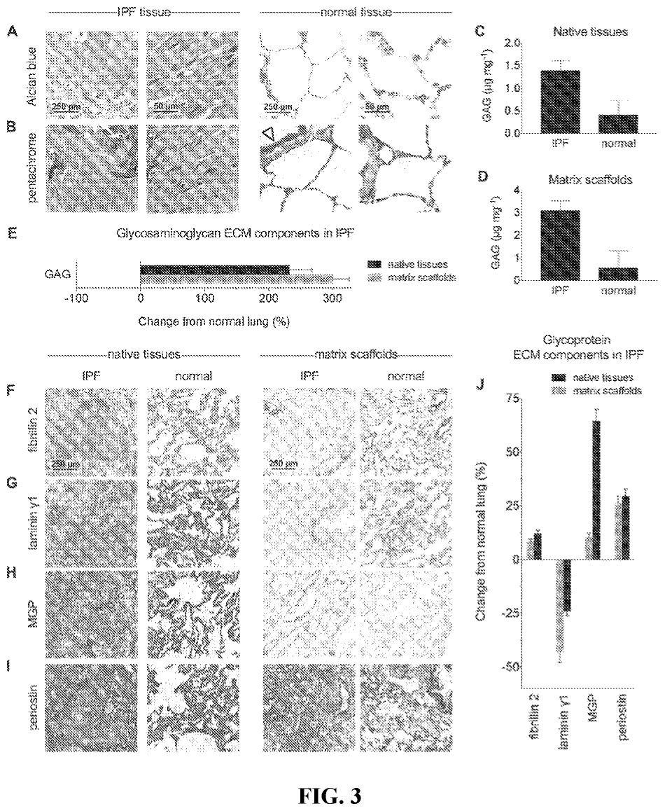

IPF and normal lung tissues and scaffolds were characterized using biochemistry. Soluble collagens were quantified in native tissues and matrix scaffolds, and increases in collagens were measured relative to normal in IPF native tissues (33.3±19.2%) and matrix scaffolds (63.2±15.6%, FIG. 2D). Consistent with the loss of elastic fibers observed in VVG elastic staining, quantification of elastin confirmed reduction in IPF native tissues (60.6±12.3%) and matrix scaffolds (54.1±17.2%) relative to normal (FIG. 2E). Altogether, the structural ECM components in IPF demonstrated clear trends relative to normal in both native tissues and matrix scaffolds: increased collagens (33-63%) and decreased elastin (54-61%; FIG. 2F). Alcian blue and pentachrome staining were performed to assess the extent and distribution of proteoglycans in IPF tissues, which was significantly higher in areas of moderate and severe fibrosis (scores≥2) than in areas of mild fibrosis (scores<2) and normal lung tissues (FIG. 3A,B). Quantification of sulfated glycosaminoglycans (GAG) revealed that GAG components in IPF native tissues and scaffolds was 232.5-300.5% higher than in normal lungs (FIG. 3C-E), consistent with overexpression of sulfated glycosaminoglycans previously observed in fibrotic foci.

Immunohistochemical staining of IPF tissues for multiple ECM glycoproteins revealed dramatic differences from normal lung tissues in fibrillin 2, laminin γ1, matrix GLA protein (MGP), and periostin (FIG. 3F-I). Areas with severe fibrosis (fibrosis score: 3) were characterized by pervasive overexpression of fibrillin 2, MGP, and periostin, and loss of laminin yl. Notably, changes from normal lung were consistent in native tissues and matrix scaffolds for all glycoproteins that were investigated (FIG. 3J).

IPF and normal lung tissues and scaffolds were characterized using proteomics. Mass spectrometry was performed on IPF and normal lung matrix scaffolds to assess the IPF matrisome (Table 1), and revealed changes from normal lung consistent with histopathologic observations and biochemical assays. Multiple collagen types increased above 150%, including collagen types I, II, V, VI, VII, XVI. Notably, in IPF lungs collagen types IV and XXI—the primary collagens comprising the alveolar basement membrane—decreased between 33-73%, consistent with the loss of basement membrane and alveolar structure associated with the progression of pulmonary fibrosis. The glycoprotein vitronectin was elevated 967%, and glycoproteins fibulin 2 and periostin were both elevated above 200%. Laminin subunits α3, β2, and γ1 and nidogen 1, which are associated with the basement membrane, were all decreased in IPF lungs. Biglycan was increased by 633%, however the basement membrane-specific heparan sulfate proteoglycan core protein was decreased by 38%. Elastin isoforms were also decreased by 31%, consistent with quantitative biochemical analyses.

Interestingly, in IPF lungs several regulators of the extracellular matrix were also increased more than 200% above normal, including metalloproteinase inhibitor 3 (TIMP3), cathepsin G, desmoplakin, and α1-antitrypsin. To assess changes in endogenous growth factors, a multiplex growth factor array was performed. Two growth factors were detected only in IPF native tissues and not in normal lung native tissues: transforming growth factor beta 3 (TGF-β3) and heparin-binding EGF-like growth factor (HB-EGF; Table 2). In IPF native tissues, insulin-like growth factor binding protein 1 (IGFBP-1) was 160-fold above normal, and both basic fibroblast growth factor (bFGF) and endocrine gland-derived vascular endothelial growth factor (EG-VEGF) were approximately 20-fold above normal. Brain-derived neurotrophic factor (BDNF) and growth differentiation factor 15 (GDF-15, a prognostic factor for IPF) were elevated 3-5-fold, but osteoprotegerin (OPG) was reduced by more than half. Five growth factors were detected in IPF matrix scaffolds (Table 3), including IGFBP-6, whose family of carrier proteins were shown to induce production of collagen type I and fibronectin in normal primary lung fibroblasts. Neurotrophin-4 (NT-4), which is elevated in explanted IPF lungs and shown to drive proliferation of primary human lung fibroblasts through TrkB-dependent and protein kinase 8-dependent pathways, was also detected in IPF matrix scaffolds.

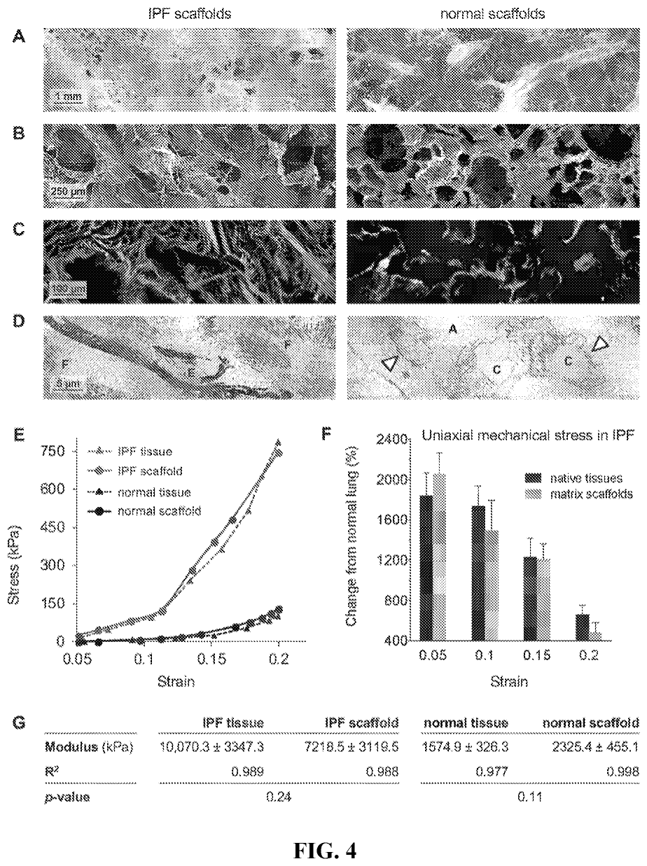

IPF matrix scaffolds were analyzed for structural, topographical, and mechanical characteristics. The gross appearance of IPF matrix scaffolds was dramatically different from the appearance of normal lung matrix scaffolds. Normal lung matrix scaffolds appeared translucent, with visible bronchial and vascular conduits and saccular structures throughout the parenchyma (FIG. 4A). By contrast, IPF matrix scaffolds had pervasive dense fibroconnective structures, with abnormal disorganized architecture, honeycombing, and no apparent airways or vessels. Scanning electron microscopy revealed dramatic disruption of normal alveolar architecture in IPF scaffolds (FIG. 4B). Topography of collagen fibers in IPF scaffolds was visualized by inverted color micrographs of trichrome staining, which showed dense fibrous bundles in IPF scaffolds and stereotypical porous (alveolar-like) networks in normal lung scaffolds (FIG. 4C). Transmission electron microscopy showed dense fibrous bands (F) of extracellular matrix in IPF matrix scaffolds with minimal evidence of normal basement membrane, whereas normal lung matrix scaffolds had abundant airspaces (A), delicate basement membrane (arrow), and alveolar capillaries (C; FIG. 4D).

Uniaxial mechanical testing of IPF and normal tissues and scaffolds indicated that IPF tissues and scaffolds were approximately 20× stiffer at 5% strain and approximately 5× stiffer at 20% strain compared to normal tissues and scaffolds (FIG. 4E). Importantly, mechanical testing also confirmed that the processing of native tissues to obtain matrix scaffolds did not alter the mechanical properties of matrix scaffolds from native tissues, as differences in elastic modulus between native tissues and matrix scaffolds were not significant (FIG. 4F,G).

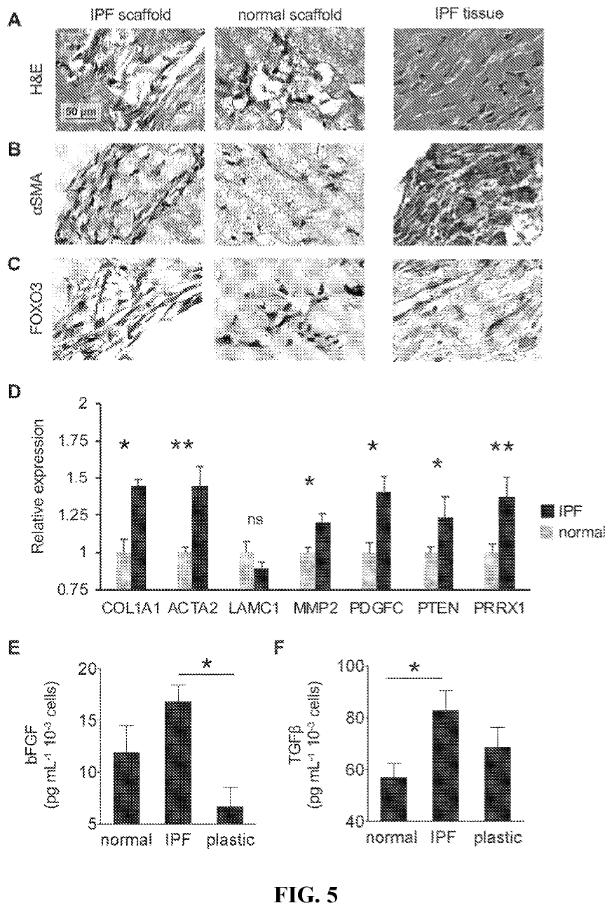

Phenotype of lung fibroblasts in IPF and normal lung scaffolds were characterized. Normal human lung fibroblasts were added to IPF and normal lung matrix scaffolds and cultured in vitro for 7 days. H&E staining showed that the phenotype of normal human lung fibroblasts varied between cells cultured in IPF and normal lung matrix scaffolds (FIG. 5A). Fibroblasts in IPF matrix scaffolds showed higher expression of alpha smooth muscle actin than fibroblasts in normal lung matrix scaffolds. Morphologic similarities between fibroblasts cultured in IPF scaffolds and IPF native tissue were observed (FIG. 5B). In contrast, immunostaining of FOXO3, a transcription factor whose downregulation is linked to fibrogenesis, showed lower expression in human lung fibroblasts cultured on IPF matrix scaffolds compared to fibroblasts cultured on normal lung matrix scaffolds (FIG. 5C). Consistent with alpha smooth muscle immunohistochemical staining, gene expression analysis showed significant upregulation of ACTA2 (alpha smooth muscle actin). Additional upregulated fibrosis-specific markers of fibroblast activation included COL1A1 (collagen type I, subunit α1), MMP2, PDGFC, PTEN, and PRRX1 (FIG. 5D). Activation of fibroblasts in vitro was also assessed by quantification of secreted basic fibroblast growth factor (bFGF) and transforming growth factor beta (TGFβ), with normal human lung fibroblasts cultured on tissue culture plastic as a standard control. Interestingly, secretion of bFGF and TGFβ were both highest with fibroblasts cultured in IPF matrix scaffolds (FIG. 5. E,F). Notably, secreted TGFβ was significantly higher in IPF matrix scaffolds compared to normal lung matrix scaffolds suggesting that substrate stiffness may have influenced secretion of TGFβ.

### EXAMPLE 2

### Lung Extracellular Matrix Hydrogels

Lung ECM was processed to yield a soluble format that can be made into hydrogel.

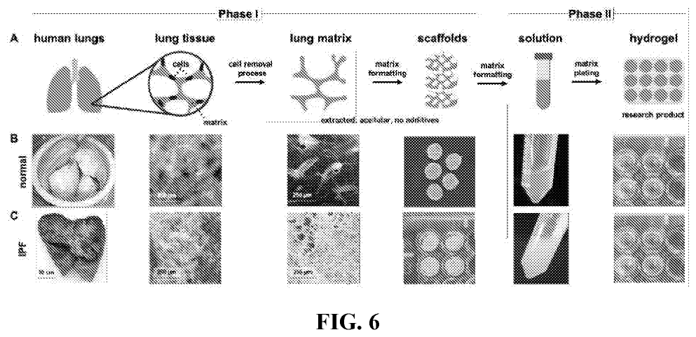

Methods: Robustly established normal and fibrotic acellular human lung matrix scaffolds were further processed to yield soluble ECM solutions that can be easily distributed and reconstituted into hydrogels in multi-well plates by addition of saline buffer or media (FIG. 6A).

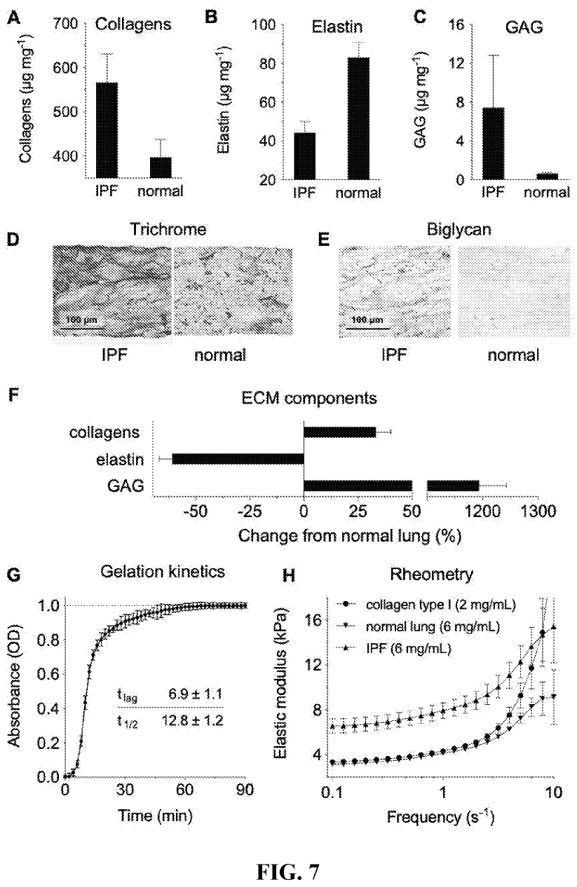

Results: Acellular normal and fibrotic lung matrix recapitulate normal and diseased lung tissue structure and histomorphology, respectively. We also established novel methods whereby lung matrix can be solubilized, dried, stored, and subsequently reconstituted into hydrogel at time of use by addition of saline buffer, media, or media with cells (FIG. 6 B,C). Normal and fibrotic lung ECM hydrogels showed concentrations of collagens (FIG. 7A), elastin (FIG. 7B), GAG (FIG. 7C) consistent with levels in intact matrix scaffolds characterized in Phase I studies, notably fibrosis-associated increased collagens and GAG, and reduced elastin in fibrotic lung ECM (FIG. 7F). Trichrome (collagens) and biglycan (proteoglycan) staining were higher in fibrotic ECM hydrogels and distributed throughout gels (FIG. 7D,E). IPF tissue stiffness is about 300% of normal lung ECM hydrogels (FIG. 7H), consistent with results in Phase I studies of native tissues and matrix scaffolds. Conclusions: Our data confirm (i) feasibility of producing lung ECM hydrogels, which (ii) maintain disease-associated biochemical and mechanical characteristics and differences between IPF and normal lung tissue.

Phenotypes of lung cells in human lung ECM hydrogels. Rationale: Disease models of lung fibrosis and cell-based drug testing platforms are increasingly complex and often utilize multiple cell types, including primary epithelial and mesenchymal cells, whose viability, cytocompatibility, and phenotype in lung hydrogels must be assessed. Phenotypes of lung cells in human lung ECM hydrogels were analyzed.

Methods: Primary human lung cells (pulmonary fibroblasts, adipose-derived mesenchymal stromal cells, bronchial and small airway epithelial cells, and alveolar basal epithelial A549 cells) were cultured in normal lung ECM (human or swine) hydrogels and competing substrates (Matrigel, collagen I, plastic) for 4-7 days, and subjected to brightfield imaging, growth assay by DNA quantification, immuno-fluorescence staining, gene expression analysis by real-time PCR, histological staining, metabolic activity assay by Alamar Blue reagent, or XTT proliferation assay.

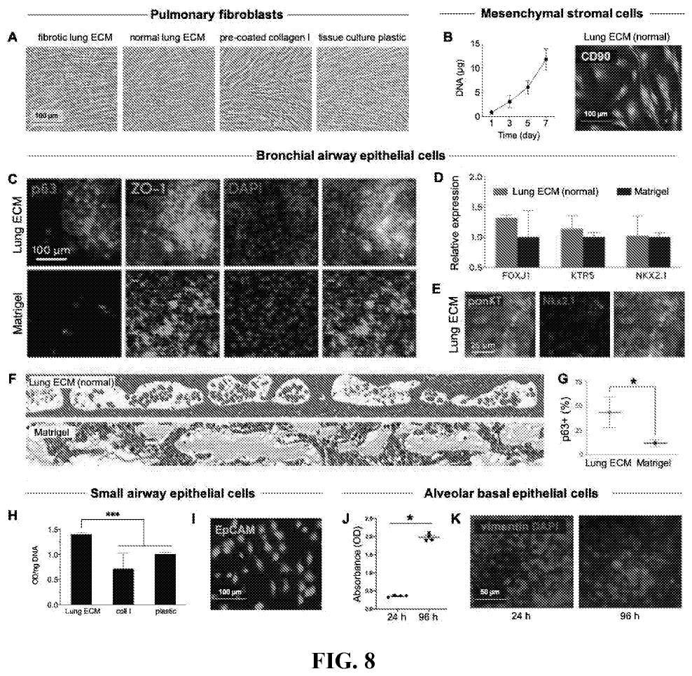

Results: Primary human pulmonary fibroblasts displayed stereotypical fibroblastic morphology (FIG. 8A). Mesenchymal stromal cells proliferated for 7 days, maintaining CD90 expression after 7 days (FIG. 8B). Bronchial airway epithelial cells in lung ECM retained significantly better phenotype of p63+ basal cells than Matrigel (FIG. 8C,G), expressed normal lung epithelial cell markers and (FIG. 8D,E), and formed larger, more robust bronchospheres in air liquid interface (ALI) cultures with lung ECM versus Matrigel (FIG. 8F). Primary human small airway epithelial cells displayed significantly higher metabolism in lung ECM hydrogel than gold standard substrates and retained EpCAM expression after 4 days (FIG. 8H,I). Alveolar basal epithelial cells (A549), a common cell model for type II pneumocytes, showed significant proliferation in lung ECM hydrogel over 4 days by XTT assay, and expressed vimentin after 4 days (FIG. 8J,K).

Conclusions: Several of the most commonly used cell types in lung fibrosis disease models and drug testing assays have robust viability, compatibility with normal lung ECM hydrogels; and in multiple assays outperform gold standard substrates including Matrigel, collagen I gel, and (collagen-coated) tissue culture plastic

### EXAMPLE 3

### Characterization of Liver Extracellular Scaffolds

Cell Removal from Human Normal and Fibrotic Liver Tissues; Analyses Biochemical Compositions of Liver Matrix Scaffolds.

Methods: Normal, fibrotic, and cirrhotic human livers were procured through approved IRB protocols, classified by histopathologic scoring, sectioned, and decellularized to obtain acellular intact normal and diseased human liver matrices, which were processed and analyzed.

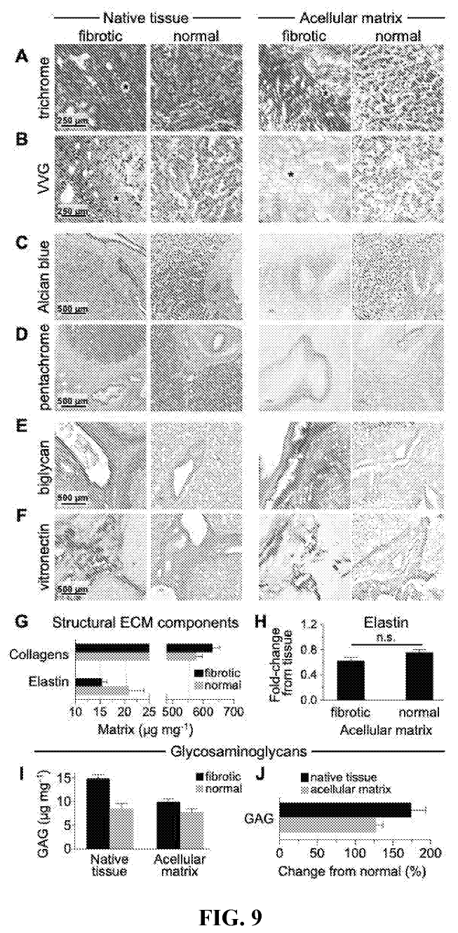

Results: Acellular human liver matrix appeared translucent, with visible sinusoidal-like conduits. Histology confirmed normal or fibrotic livers. H&E staining revealed drastic differences in the architecture of normal and fibrotic liver matrix scaffolds, with no discernible nuclei. Removal of >99.5% nuclear material after tissue processing was confirmed by DNA assay. Normal and fibrotic liver matrix scaffolds showed pathological histomorphology consistent with liver tissues. Trichrome stain showed dramatic deposition of collagens in densely aligned bundles throughout regions of severe fibrosis in fibrotic native tissue and acellular matrix (FIG. 9A). Verhoeff-Van Gieson (VVG) elastic staining showed a noticeable loss of elastic fibers (black) in fibrotic regions, whereas elastic fibers were dispersed more homogenously throughout the normal parenchyma (FIG. 9B). Liver matrix scaffolds retain liver fibrosis-specific structure, biochemical composition, and glycoprotein distribution. Alcian blue and pentachrome staining showed distribution of proteoglycans in fibrotic tissues was significantly higher in areas of moderate and severe fibrosis (scores≥3) than in areas of mild fibrosis (scores<2) and normal liver tissues (FIG. 9C-D). Immunohistochemical staining for glycoproteins revealed dramatic differences from normal liver tissue in biglycan (FIG. 9E) and vitronectin (FIG. 9F). Biochemical analysis of normal and fibrotic liver ECM showed concentrations of collagens (FIG. 9G) and elastin (FIG. 9H) consistent with histological observations. Sulfated glycosaminoglycans (GAG) components in fibrotic tissues and matrix were 157-193% and 119-137% higher, respectively, than normal (FIG. 9I-J), consistent with overexpression of sulfated glycosaminoglycans observed in fibrotic foci. To assess the matrisome of fibrotic and cirrhotic livers, mass spectrometry was performed and revealed a growing trend of deposition of ECM components in mild fibrotic and cirrhotic livers compared to normal liver consistent with histopathologic observations and biochemical assays (Table 3).

Conclusions: Normal fibrotic and cirrhotic human liver matrix scaffolds were cell-free with high preservation of architecture, biochemical composition, and topography similar to human liver tissues. Our preliminary data support the value proposition for users seeking highly physiological cell culture environments replicating the human liver extracellular matrix of alcoholic liver disease, and form a robust basis for deeper compositional analyses (proteomics) and in-vitro applications (cell-based assays) of human liver scaffolds.

### Biocompatibility and Comparative Function of Human Liver Cell Types in Liver ECM Scaffolds Versus Competing Substrates.

Liver-specific ECM substrates (normal or fibrotic) offer significant advantages over available substrates.

Methods: Primary human hepatocytes, HepG2 cells (human hepatocarcinoma cell type commonly used to model hepatocytes), and human hepatic stellate cells (HSC) were cultured for up to 7 days on normal or fibrotic human liver matrix scaffolds, Matrigel, collagen I hydrogels, and/or tissue culture plates (plastic) with or without collagen I coating.

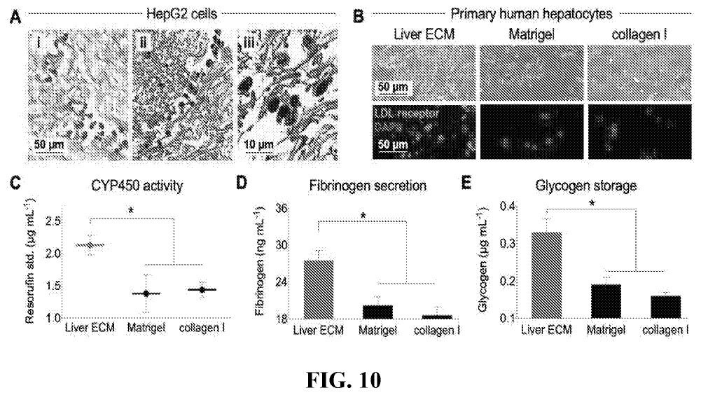

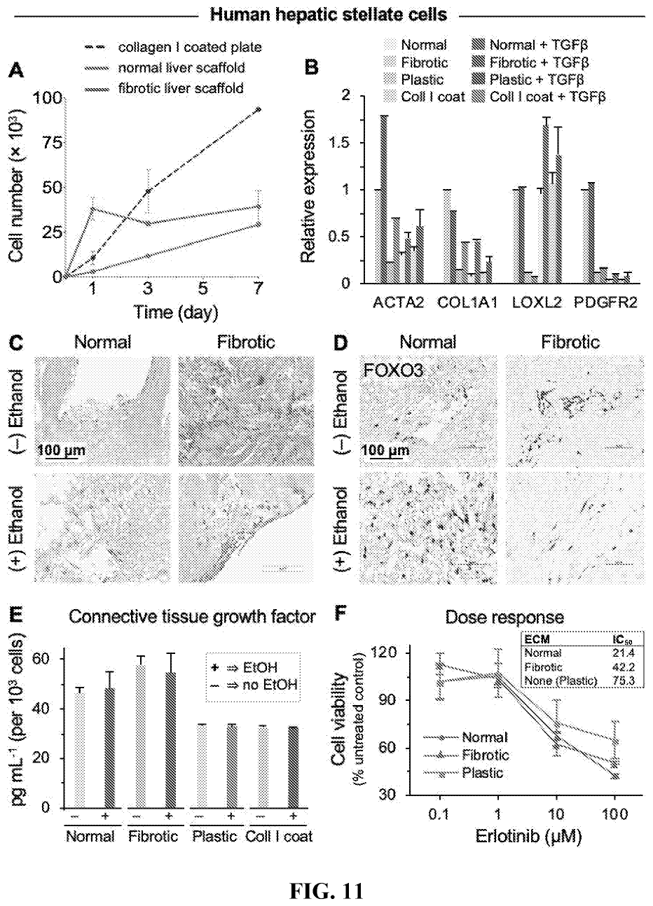

Results: In vitro, liver matrix scaffolds supported robust liver cell adhesion, viability (FIG. 10A), structure formation, significantly higher LDL uptake (FIG. 10B), cytochrome P450 (CYP1A2) activity (FIG. 10C), fibrinogen secretion (FIG. 10D), glycogen storage (FIG. 10E), compared to Matrigel and collagen I hydrogel. HSCs integrated into 3D liver matrix scaffolds exhibited different proliferation rate on normal, and fibrotic ECM scaffolds (FIG. 11A) with notably higher proliferation in fibrotic scaffolds than normal scaffolds, consistent with HSC activity in progressive hepatofibrotic disease. HSCs differentially expressed fibrosis-related genes ACTA2, COL1A1 LOXL2 and PDGFR2 in liver scaffolds versus tissue plastic and Coll coated plates (FIG. 11B). Controversially HSC on ECM scaffolds exhibit attenuated response to TGFβ addition to the media. Although morphologic similarities were observed between HSC cultured in fibrotic and normal liver ECM scaffolds, morphology differed from HSC cultured on artificial substrates (FIG. 11C). Furthermore, immunostaining for FOXO3 (FIG. 11D), a transcription factor whose downregulation is linked to fibrogenesis, showed lower expression and translocation to the cytoplasm in HSC cultured in fibrotic liver ECM scaffolds compared to HSC cultured in normal liver ECM scaffolds, even when HSC on normal scaffold were treated with EtOH. HSC on liver scaffolds showed elevated secretion of connective tissue growth factor (FIG. 11E), a major mediator of tissue remodeling and fibrosis, compared to plastic grown cells. To test whether HSC cultured on different ECM respond differently to drug treatment then cells grown in standard 2D we measured the IC50 response of HSC to Erlotinib, an inhibitor of the epidermal growth factor receptor (EGFR) tyrosine, used for the treatment for several cancers and currently is in clinical trials for the treatment of hepatocarcinomatous originating from cirrhotic livers (FIG. 11F). 5000 cells were plated on 96 wells on fibrotic or normal ECM soluble ECM and on plastic. 24 hours later Erlotinib or DMSO vehicle in different concentrations was added. Cells were grown with drugs for 72 hours (with media+drug replenished every day). Cell number were assed using a standard XTT assay. IC50 values were lower for cells on normal ECM compared to fibrotic ECM and higher for plastic cultured cells.

Conclusions: Human liver matrix scaffolds supported superior viability, growth, multiple cell-specific phenotype/functions of multiple human liver cell types, including primary human hepatocytes and hepatic stellate cells, putative effector cells of liver fibrosis.

Summary: Tissue-derived fibrotic and normal liver ECM scaffolds have great potential to serve as highly patho/physiologically relevant in-vitro cell culture substrates for chronic liver disease R&D and anti-fibrotic drug development. Our preliminary studies demonstrate that fibrotic and normal liver matrix scaffolds have been successfully developed and initially characterized by: (i) histology, (ii) biochemical and ultrastructural properties (FIG. 9), (iii) high biocompatibility, supporting a diversity of hepatocellular functions, and superior performance against competing substrates to provide human liver cells (primary/lines) a human liver-specific environment in vitro (FIG. 10&11).

### EXAMPLE 4

### Characterization of Liver Extracellular Scaffolds

Determination of primary molecular components and biochemical properties of human fibrotic liver extracellular matrix.

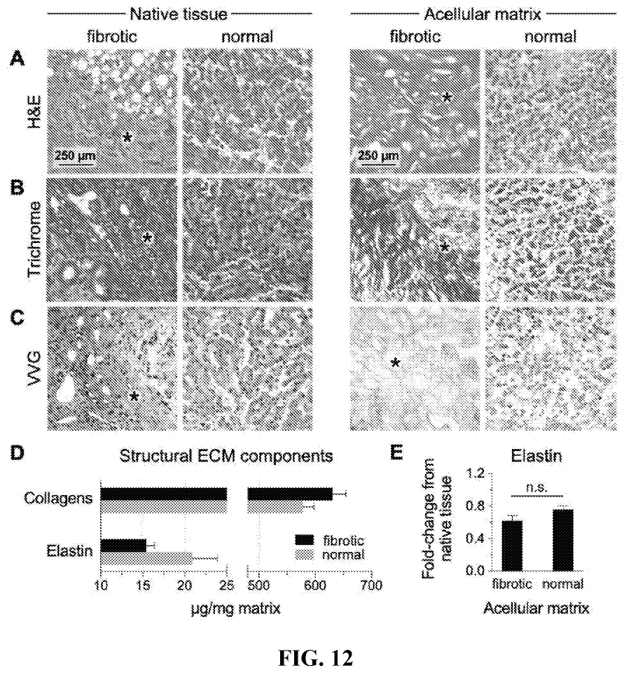

Methods: Human native liver tissues were treated to remove cellular and nuclear components, which was confirmed by hematoxylin and eosin staining (FIG. 12A) and quantitative DNA assay, which confirmed that >98% DNA content of native tissues was removed. The resulting intact ECM (often referred to as ‘scaffolds’) was then further processed to yield soluble ECM preparations that can be readily reconstituted into hydrogels in multi-well plates by addition of saline buffer or media with or without cells. ECM from human livers were confirmed negative for mycoplasma, bacteria, and fungi and deemed suitable for use in in-vitro studies.

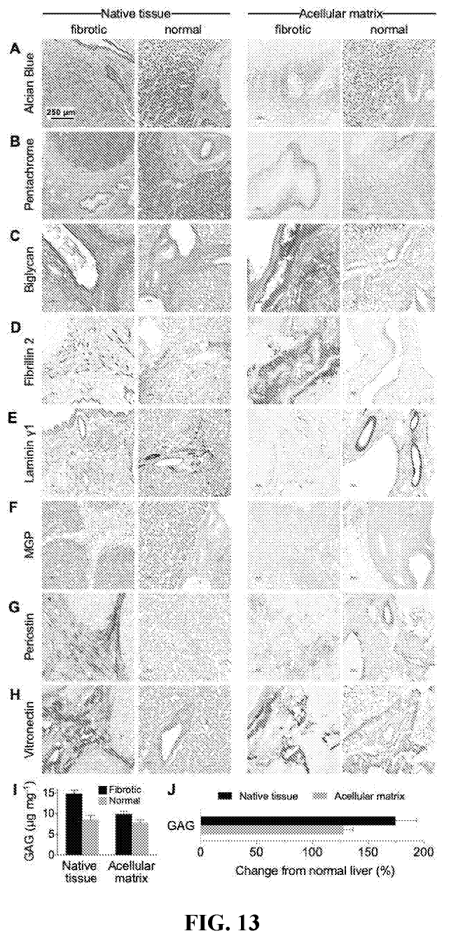

Results: Acellular normal and fibrotic liver matrix recapitulate normal and disease-specific histologic features. For histologic evaluations of liver fibrosis, representative fields corresponding to fibrosis scores ≥F3 (severe fibrosis) were imaged. To visualize distributions of ECM structural components in fibrotic and normal livers, histologic staining was performed on native (untreated) tissues and acellular matrix (intact ECM after cell removal). H&E staining of native fibrotic liver tissues revealed numerous regions with severe distortion of liver structure and bridging fibrous septa, especially around blood vessels and biliary structures (FIG. 12A, star indicates representative region with severe fibrosis, excessive deposition of collagens, and loss of elastic fibers). In contrast, H&E staining of native normal liver tissues displayed regular sinusoidal structure and minimal or no steatosis. Acellular matrix from analogous regions of fibrotic and normal livers had no discernible nuclei and displayed drastic differences in ECM architecture consistent with fibrotic and normal native liver tissues, respectively. Trichrome staining showed dramatic deposition of collagens (blue) in densely aligned bundles throughout regions of severe fibrosis (FIG. 12B) in fibrotic native tissue and acellular matrix. Verhoeff-Van Gieson (VVG) elastic staining showed a noticeable loss of elastic fibers (black) in fibrotic regions, whereas elastic fibers were dispersed more homogenously throughout the normal parenchyma (FIG. 12C). Normal and fibrotic liver ECM hydrogels showed concentrations of collagens (FIG. 12D) and elastin (FIG. 12D,E) consistent with respective histological characterizations. Alcian blue and pentachrome staining were performed to assess extent and distribution of proteoglycans in fibrotic tissues, which was significantly higher in areas of moderate and severe fibrosis (scores≥3) than in areas of mild fibrosis (scores<2) and normal liver tissues, consistent with overexpression of sulfated glycosaminoglycans previously observed in fibrotic foci. Immunohistochemical staining of human fibrotic liver tissues for multiple glycoproteins revealed dramatic differences from normal liver tissue in biglycan, fibrillin 2, laminin γ1, matrix GLA protein (MGP), periostin, and vitronectin (FIG. 13C-H). Quantification of sulfated glycosaminoglycans (GAG) consistently revealed that GAG components in fibrotic native tissues and acellular matrix was 157-193% and 119-137% higher, respectively, than normal (FIG. 13I,J). Mass spectrometry was performed on fibrotic and normal liver ECM to assess the fibrotic liver matrisome (Table 4), and revealed numerous changes from normal liver consistent with histopathologic observations and biochemical assays. Multiple collagen types increased above 150%, including collagen types I, II, VI, VII, XII, XIV. Notably, Collagen VI is associated with increased matrix deposition in foci of severe perisinusoidal fibrosis, fibrotic portal tracts, and fibrous septa. Consistent with increased expression observed in histological and biochemical analyses, several glycoproteins and proteoglycans exhibited elevated expression in fibrotic ECM compared to normal. Multiple laminin subunits showed elevations over 200%, and glycoproteins fibulin 2, periostin, and fibronectin were elevated 7-to 20-fold. Notably, biglycan was increased by 700%, and TIMP3, a major ECM regulator, was decreased by 48%.

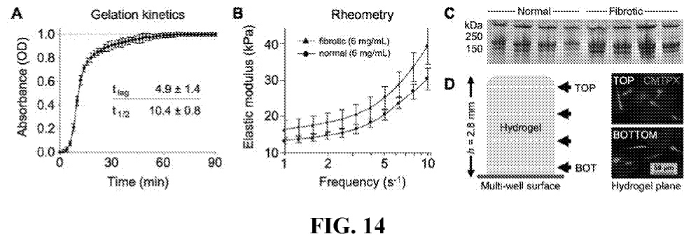

Physiochemical characterizations of fibrotic and normal human liver matrix hydrogels. Human liver ECM hydrogels exhibited gelation kinetics with stereotypical sigmoidal curves, t_{lag}˜5 min, and t_{1/2}˜10 min (FIG. 14A), acceptable gelation times according to current beta product user feedbacks and prospective user interviews. Rheometric testing of fibrotic and normal liver ECM hydrogels revealed that fibrotic ECM hydrogel is approximately 1.5× stiffer at 5-10% strain, which is a similar change to values reported for changes in fibrotic tissues (FIG. 14B). Protein size distributions visualized by SDS PAGE showed consistent differences between fibrotic and normal ECM from different donors (FIG. 14C), suggesting common or stereotypical ECM components across donors and disease progression. To confirm the utility of ECM hydrogels for drug testing compounds up to molecular weight 800 g mol⁻¹ (large molecules, antibodies, growth factors, imaging probes, etc.), we verified diffusivity of multiple fluorescent trackers through liver ECM hydrogels, and confirmed CellTracker Red CMTPX had a diffusion rate>2 mm h⁻¹ (FIG. 14D). Conclusions: Our data confirm: (i) feasibility of producing liver ECM hydrogels that (ii) maintain disease-associated biochemical and mechanical profiles and characteristics of fibrotic and normal liver tissues.

### EXAMPLE 5

### Demonstration of Anti-Fibrotic Therapeutics on Fibrotic Lung ECM Scaffolds

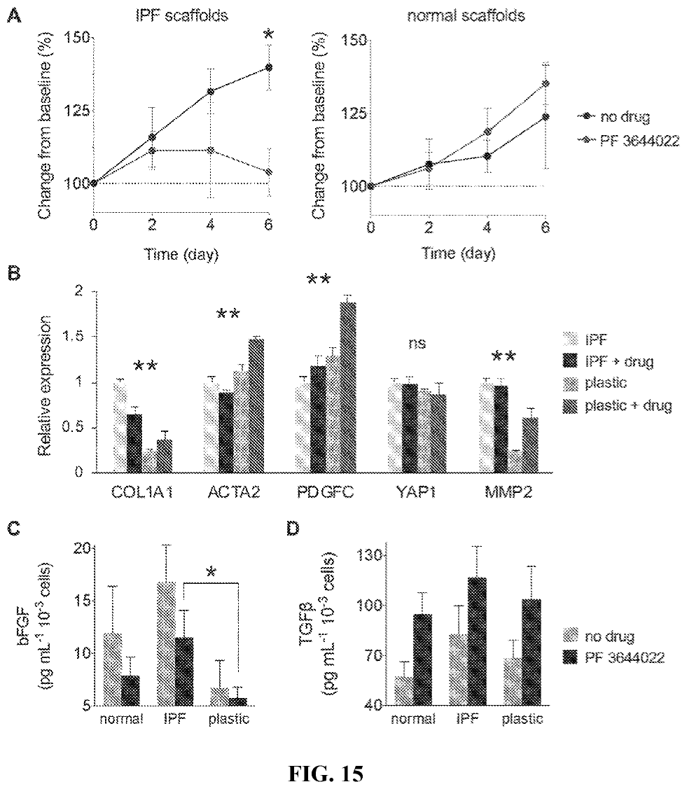

Growth curves of pulmonary fibroblasts cultured in IPF and normal lung matrix scaffolds and exposed to antifibrotic agent PF3644022 (an ATP-competitive MK2 inhibitor) demonstrated significantly different profiles (FIG. 15A). In the absence of antifibrotic drug, over 6 days, fibroblasts in IPF matrix scaffolds had a mean growth rate (linear fit: slope=6.74, R2=0.98) over 80% faster than fibroblasts in normal lung matrix scaffolds (linear fit: slope=3.70, R2=0.93). In the presence of antifibrotic drug, fibroblasts in IPF matrix scaffolds demonstrated greater sensitivity and drug response than fibroblasts in lung matrix scaffolds, whose growth was not significantly different from untreated cells after 6 days. Gene expression varied significantly between fibroblasts cultured on IPF matrix scaffolds and plastic. Interestingly, expression of COL1A1 and MMP2 by fibroblasts cultured on tissue culture plastic was lower than expression by fibroblasts in IPF matrix scaffolds, suggesting that the presence of disease-specific matrix results in a fibroblast phenotype in vitro consistent the diseased phenotype in humans (FIG. 15B). In IPF matrix scaffolds, treatment with antifibrotic agent PF3644022 consistently reduced expression of COL1A1 and ACTA2. No significant differences were observed in the expression of YAP1. Similar trends were observed in secretion of bFGF (FIG. 15C) and TGFβ (FIG. 15D) by fibroblasts exposed to the antifibrotic agent.

In this study, we demonstrated the use of IPF disease-specific ECM in a 30 cell-based assay of antifibrotic agent PF3644022 (an MK2 inhibitor in IPF model). As expected, fibroblasts cultured on fibrotic lung ECM scaffolds and treated with PF3644022 exhibited greater sensitivity and drug response, significantly different gene expression, and downregulation of genes associated with ECM production compared to cells cultured on tissue culture plastic. We envision that disease-specific ECM may be applicable across multiple stages of the early-stage drug discovery pipeline, from target selection and hit identification through lead identification and optimization. The use of disease-specific ECM substrates is consistent with the set of principles defined for ‘disease-relevant assays’ that specifically recommend ensuring: (i) substrate tension and mechanical forces are appropriate, and (ii) extracellular matrix composition is relevant, with appropriate tissue architecture, cell differentiation and function to enhance clinical translation of the in-vitro assay. Ultimately, implementation of disease-specific ECM components or substrates into preclinical human disease models and cell-based screening assays could increase clinical relevance and success rates.

### EXAMPLE 6

### Method of Making Liver Extracellular Matrix Scaffolds

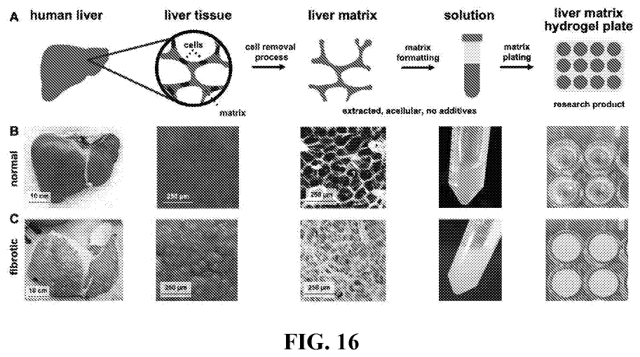

Decellularized liver extracellular matrix can be formulated into multiple different end products, such as coating materials, hydrogels or 3D scaffolds. The first step in creating any of these products is to identify diseased liver tissue and collect tissue samples. The tissue from these samples then undergoes a cell removal process, or decellularization, and the extracellular matrix is them isolated away from the rest of the cellular components and can be processed into the coating material, hydrogel, 3D scaffold or more. The products can be utilized in a number of ways, but can be used to coat cell-culture plates and used in research to investigate the effects of diseased ECM on human cells or to test the efficacy of therapeutics or disease models (FIG. 16).

### EXAMPLE 7

### Fibrotic Liver Extracellular Scaffolds Co-cultures with Primary Hepatic Stellate Cells

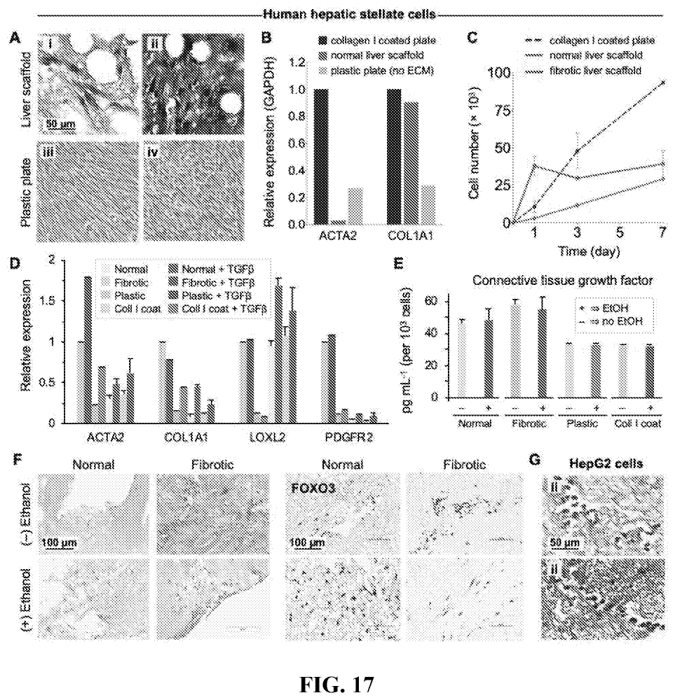

Assessment of intact ECM (scaffolds) supporting in-vitro fibrosis modeling: HSC were cultured in liver ECM scaffolds for 5-7 days, and on tissue cultured plastic and collagen I coated plates as controls. Cells proliferated slower on liver ECM scaffolds than on collagen I coated plates (FIG. 17C). Gene expression analysis showed significant upregulation of ACTA2 (alpha smooth muscle actin) in cells on plastic compared to cells in liver ECM, indicating reduced activation in normal liver ECM scaffolds, notably with or without supplemental TGFβ (FIG. 17D). Additional upregulated fibrosis-specific markers of fibroblast activation included COL1A1 (collagen type I, subunit al) and PDGFR2. Expression of LOXL2 was higher in cells cultured in both normal and fibrotic liver ECM. We also compared HSC secretory function in response to ethanol by measuring the level of connective tissue growth factor (CTGF) by enzyme linked immunosorbent assay (ELISA) and observed higher secretion in cells cultured in ECM (FIG. 17E). Although morphologic similarities were observed between HSC cultured in fibrotic and normal liver ECM scaffolds, morphology differed from HSC cultured on artificial substrates (FIG. 17A,F). Furthermore, immunostaining for FOXO3, a transcription factor whose downregulation is linked to fibrogenesis, showed lower expression and translocation to the cytoplasm in HSC cultured in fibrotic liver ECM scaffolds compared to HSC cultured in normal liver ECM scaffolds. In conclusion, we confirmed the compatibility of normal & fibrotic liver ECM scaffolds with common analytical techniques & assays used in modelling early-stage drug development including discovery, screening, target validation.

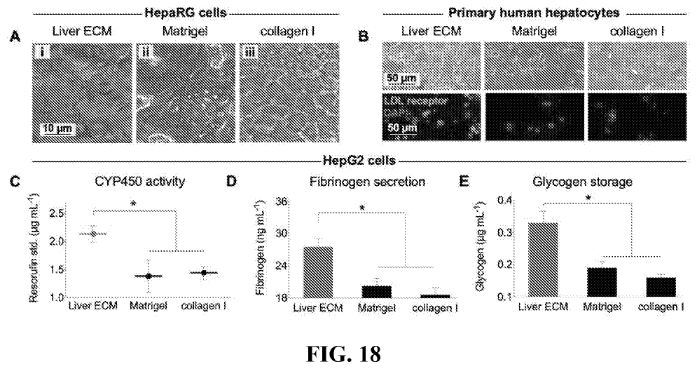

Testing hepatocyte cultures on ECM hydrogels: Disease models of liver fibrosis and cell-based drug testing platforms are increasingly complex and often utilize multiple cell types, including primary hepatocytes, Hepg2 cells, Kupfer cells and sinusoidal endothelial cells whose viability, cytocompatibility, and functionality in liver hydrogels must be assessed. Primary human hepatocytes or Hepg2 cells were cultured in normal liver ECM (human or swine) hydrogels and competing substrates (Matrigel, collagen I, plastic) for 4-7 days, and subjected to brightfield imaging, immuno-fluorescence staining and functional assays. Hepatocytes cultured on 3D normal swine liver ECM hydrogels for 7 days exhibit the formation of 3D structures (FIG. 18A, top) express higher expression of low-density lipoprotein receptor (FIG. 18A, bottom). Cytochrome P450 (CYP1A2) activity (FIG. 18B), fibrinogen secretion (FIG. 18C), and glycogen storage (FIG. 18D), were significantly elevated in cells grown on liver ECM compared to Matrigel and collagen I gel (*p<0.01). In conclusion, several of the most commonly used cell types in liver fibrosis disease models and drug testing assays have robust viability, compatibility and enhanced functionality when grown on normal liver ECM hydrogels; and in multiple assays outperform gold standard substrates including Matrigel, collagen I gel, and (collagen-coated) tissue culture plastic.

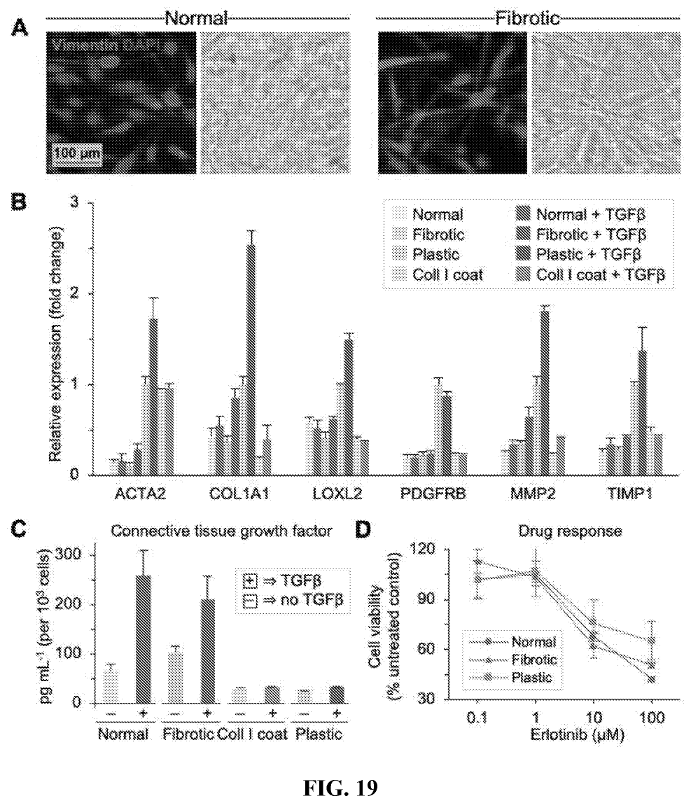

Compatibility and the advantages of soluble fibrotic ECM for modeling fibrosis in-vitro: Demonstrating compatibility of fibrotic human liver ECM hydrogel for modeling liver fibrosis using standard techniques and drug testing. Normal human hepatic stellate cells were added to fibrotic & normal liver soluble ECM hydrogel and cultured in vitro for up to 7 days, showing expression of typical markers like vimentin and KI67 using high-power micrographs (FIG. 19A). Gene expression analysis of HSC grown in fibrotic ECM with or without TGFβ, showed higher expression of alpha smooth muscle actin than HSC cultured in normal liver ECM (FIG. 19B; cultured in liver matrix hydrogels “fibrotic”, collagen I coating “coll I coat”, and tissue culture plastic “plastic”). Additional upregulated fibrosis-specific markers of fibroblast activation included COL1A1 (collagen type I, subunit al), MMP2, PDGFC, and TIMP1 (FIG. 19B). However, all genes except LOXL2 showed higher expression on plastic and collagen 1 coated compared to HSC grown on ECM. Functionality of cells in-vitro was also assessed by quantification of secreted connective tissue growth factor (CTGF) and procollagen 1 by HSC cultured on tissue culture plastic as a standard control. Interestingly, secretion of CTGF was highest with HSC cultured on ECM as measured by ELISA (FIG. 19C). Notably, without TGFβ addition secreted CTGF was significantly higher in cells grown on fibrotic ECM compared to normal liver matrix, suggesting that substrate stiffness may have influenced secretion of CTGF. To further test the feasibility of the hydrogel system for testing drug function we measured the IC50 response of HSC to Erlotinib, an inhibitor of the epidermal growth factor receptor (EGFR) tyrosine, used for the treatment for several cancers and currently is in clinical trials for the treatment of hepatocarcinomatous originating from cirrhotic livers. 5000 cells were plated on 96 wells on fibrotic or normal ECM hydrogel and on plastic. 24 hours later Erlotinib or DMSO vehicle in different concentrations was added. Cells were grown with drugs for 72 hours (with media +drug replenished every day). Cell number were assed using a standard XTT assay (FIG. 19D).

### EXAMPLE 8

### Drug Response Assay Using Liver ECM Hydrogels

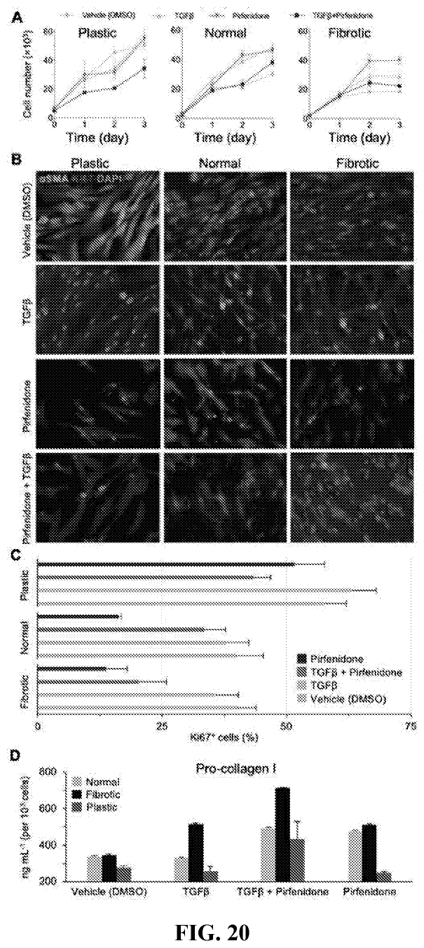

Compatibility with analytical techniques and assays used in drug development: Demonstrating compatibility of fibrotic human liver ECM hydrogel with techniques used in HTS applications will open significant market opportunities in anti-fibrotic drug testing. Normal HSC were cultured in fibrotic human liver ECM hydrogel for up to 5 days. Cells were cultured in fibrotic liver ECM hydrogel for 2 days, then TGFβ1 (25 nM) was added to induce activation, along with 2.7 mM Pirfenidone (FDA-approved gold standard anti-fibrotic drug) or DMSO vehicle (1%) control for 3 days prior to assay readouts on day 5. Cell-based proliferation assay, immunofluorescence imaging in multi-well plates, and Elisa for ProCollagen1 were performed. These studies demonstrated that addition of TGFβ accelerated cell proliferation, while Pirfenidone slowed cell proliferation (FIG. 20A). Compatibility of fibrotic liver ECM hydrogel with a standard imaging for anti-fibrotic drug was demonstrated: addition of TGFβ significantly changed the percentage of proliferating Ki67+ cells measured (FIG. 20B,C), while Pirfenidone significantly reduced the number of Ki67 positive cells demonstrating an application of the fibrotic human liver ECM hydrogel to achieve anti-fibrotic effect of Pirfenidone to decrease proliferation of HSC in vitro. Notably Pirfenidone affect was more dramatic on liver ECM compared to plastic (˜20% Ki67 positive cells on ECM compared to over 50% in plastic after Pirfenidone treatment). Conclusions: We confirmed compatibility of normal & fibrotic liver ECM hydrogel with common analytical techniques & assays used in early-stage drug development including discovery, screening, target validation.

### EXAMPLE 9

### Drug Response Assay Using Lung ECM Hydrogels

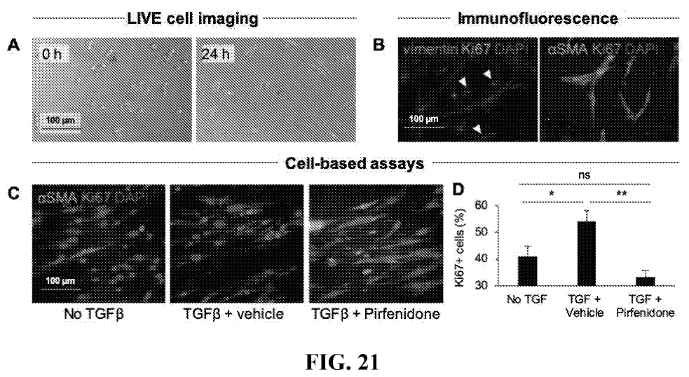

Compatibility with analytical techniques and assays used in drug development: Demonstrating compatibility of fibrotic human lung ECM hydrogel with techniques used in HTS applications will open significant market opportunities in anti-fibrotic drug testing. Normal human pulmonary fibroblasts were cultured in fibrotic human lung ECM hydrogel for up to 5 days and subjected to live cell imaging with CellTracker™ red, immunofluorescence imaging in multi-well plates, and a common cell-based proliferation assay. Cells were cultured in fibrotic lung ECM hydrogel for 2 days, then TGFβ1 (5 nM) was added to induce myofibroblast differentiation, along with 2.7 mM Pirfenidone (FDA-approved gold standard anti-fibrotic drug) or DMSO vehicle (10%) control for 2 days prior to assay readouts on day 5. Results: Live cell imaging revealed human lung fibroblasts had round morphology immediately after embedding (0 h) in hydrogel but developed stereotypical spindle morphology by 24 hours (FIG. 21A), indicating adhesion to fibrotic lung ECM hydrogel. After 48 hours, 1standard immunofluorescence staining was performed in situ in 96-well plates. Fibroblasts expressed vimentin, αSMA, and proliferation marker Ki67 (white arrows, FIG. 21B). Compatibility of fibrotic lung ECM hydrogel with a standard cell-based proliferation assay for anti-fibrotic drug testing was also demonstrated: addition of TGFβ increased αSMA and significant changes in percentage of proliferating Ki67+ cells were measured (FIG. 21C,D), demonstrating an application of the fibrotic human lung ECM hydrogel to achieve the appropriate myofibroblast differentiation and anti-fibrotic effect of Pirfenidone to decrease proliferation of lung fibroblasts in vitro. In conclusion, these results confirm compatibility of normal & fibrotic lung ECM hydrogel with common analytical techniques & assays used in early-stage drug development including discovery, screening, target validation.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. An in vitro cell culture substrate comprising:
an acellular tissue-specific extracellular matrix derived from a fibrotic tissue, wherein the tissue-specific extracellular matrix comprises fragmented macromolecules.

2. The substrate of claim 1, wherein the substrate is one of a hydrogel, a surface coating, a scaffold, a bio-ink, a media supplement, and a sponge.

3. The substrate of claim 1, wherein the fragmented macromolecules comprise collagens, glycoproteins, proteoglycans, laminins, extracellular matrix associate proteins, soluble growth factors, inflammatory cytokines, and immune mediators.

4. The substrate of claim 3, wherein a concentration of the collagens is elevated with respect to healthy, non-fibrotic tissue.

5. The substrate of claim 3, wherein a concentration of the glycoproteins is elevated with respect to healthy, non-fibrotic tissue.

6. The substrate of claim 3, wherein a concentration of the laminins is elevated with respect to healthy, non-fibrotic tissue.

7. The substrate of claim 3, wherein a concentration of the elastins is reduced with respect to healthy, non-fibrotic tissue.

8. The substrate of claim 1, wherein the fibrotic tissue is tissue exhibiting pulmonary fibrosis.

9. The substrate of claim 8, wherein the fragmented macromolecules comprise collagens or subunits thereof in a concentration of about 100 μg/mL to about 400 μg/mL.

10. The substrate of claim 9, wherein the collagens comprise:
an elevated concentration with respect to healthy, non-fibrotic lung tissue of at least one of collagen type II α1 chain and collagen type XVI α1 chain; and
a reduced concentration with respect to healthy, non-fibrotic lung tissue of at least one of collagen type IV α1 chain, collagen type IV α2 chain, collagen type IV α3 chain, collagen type IV α4 chain, collagen type IV α5 chain, and collagen type XXI α1 chain.

11. The substrate of claim 8, wherein the tissue-specific extracellular matrix further comprises:
an elevated concentration with respect to healthy, non-fibrotic lung tissue of at least one of fibulin 2, periostin, vitronectin, and laminin α5; and
a reduced concentration with respect to healthy, non-fibrotic lung tissue of at least one of laminin γ1, laminin β2, nidogen 1, and laminin α3.

12. The substrate of claim 8, wherein the tissue-specific extracellular matrix further comprises an elevated concentration with respect to healthy, non-fibrotic lung tissue of at least one of growth differentiation factor 15, brain-derived neurotrophic factor, insulin-like growth factor binding protein 6, and hepatocyte growth factor.

13. The substrate of claim 8, wherein the tissue-specific extracellular matrix further comprises a plurality of growth factors comprising:
transforming growth factor β3 at a concentration of at least 10 pg/mL;
heparin-binding EGF-like growth factor at a concentration of at least 1 pg/mL;
basic fibroblast growth factor at a concentration of at least 100 pg/mL; and
growth differentiation factor 15 at a concentration of at least 100 pg/mL.

14. The substrate of claim 8, wherein the substrate comprises an elastic modulus of at least about 20 kPa.

15. The substrate of claim 1, wherein the fibrotic tissue is tissue exhibiting liver fibrosis.

16. The substrate of claim 15, wherein the fragmented macromolecules comprise collagens or subunits thereof in a concentration of about 500 μg/mg to about 700 μg/mg.

17. The substrate of claim 15, wherein the collagens comprise:
an elevated concentration with respect to healthy, non-fibrotic liver tissue of at least one of collagen type XIV al chain and collagen type XII al chain; and
a reduced concentration with respect to healthy, non-fibrotic liver tissue of at least one of collagen type IV α1 chain and collagen type VI α6 chain.

18. The substrate of claim 15, wherein the tissue-specific extracellular matrix further comprises an elevated concentration with respect to healthy, non-fibrotic liver tissue of at least one of transforming growth factor β3, laminin β1, periostin, and fibronectin.

19. The substrate of claim 15, wherein the substrate comprises an elastic modulus of at least about 15 kPa.

20. A method of assessing an in vitro fibrotic cell culture, the method comprising:
providing one or more substrates comprising an acellular tissue-specific extracellular matrix comprising fragmented macromolecules derived from fibrotic tissue, wherein each substrate is provided in segregated manner;
culturing native cells in each substrate to form a fibrotic cell culture; and
assessing at least one characteristic of each fibrotic cell culture.

21. The method of claim 20, further comprising:
contacting each fibrotic cell culture with a drug,
wherein the at least one characteristic comprises a response to the drug.

22. The method of claim 20, wherein the at least one characteristic comprises one or more of a gene expression profile, a protein expression profile, cell proliferation, extracellular matrix interaction, cell differentiation, cell migration, cell viability, cell, cell metabolism, and cell invasion.

23. A method of assessing a drug response of a fibrotic cell culture, the method comprising:
providing one or more first substrates comprising an acellular tissue-specific extracellular matrix comprising fragmented macromolecules derived from fibrotic tissue, wherein each first substrate is provided in a segregated manner;
providing one or more second substrates comprising an acellular tissue-specific extracellular matrix comprising fragmented macromolecules derived from non-fibrotic tissue, wherein each second substrate is provided in a segregated manner;
culturing native cells in each first substrate to form a fibrotic cell culture;
culturing native cells in each second substrate to form a non-fibrotic cell culture;
contacting each fibrotic cell culture and each non-fibrotic cell culture with a drug; and
assessing a response of each fibrotic cell culture and each non-fibrotic cell culture to the drug.

24. The method of claim 23, wherein the at least one characteristic comprises one or more of a gene expression profile, a protein expression profile, cell proliferation, extracellular matrix interaction, cell differentiation, cell migration, cell viability, cell, cell metabolism, and cell invasion.

25. A kit for constructing a plurality of fibrotic tissue substrates, the kit comprising:
one or more substrate precursors, each substrate precursor comprising a different acellular tissue-specific extracellular matrix comprising fragmented macromolecules derived from fibrotic tissue; and
at least one reagent configured to convert each substrate precursor into a tissue-specific extracellular matrix substrate.

26. An in vitro cell culture substrate comprising:
a deconstructed matrisome including one or more fragmented macromolecules derived from a fibrotic tissue, wherein the one or more fragmented macromolecules comprise collagens, glycoproteins, proteoglycans, laminins, extracellular matrix associate proteins, soluble growth factors, inflammatory cytokines, and immune mediators,
wherein (i) a concentration of each of the collagens, the glycoproteins, and the laminins is elevated with respect to healthy, non-fibrotic tissue, and (ii) a concentration of the elastins is reduced with respect to healthy, non-fibrotic tissue,
wherein the substrate comprises an elastic modulus of at least about 15 kPa, and
wherein the substrate lacks an extracellular ultrastructure of the fibrotic tissue.
